(19) United States
(12) Patent Application Publication
(10) Pub. No.: US 2018/0184962 A1
(43) Pub. Date: Jul. 5, 2018

(54) METHODS AND SYSTEMS FOR DETERMINATION OF MENTAL STATE

(71) Applicant: BrainMarc Ltd., Yokneam Illit (IL)

(72) Inventor: Goded Shahaf, Haifa (IL)

(73) Assignee: BrainMarc Ltd., Yokneam Illit (IL)

(21) Appl. No.: 15/739,289

(22) PCT Filed: Jun. 23, 2016
(86) PCT No.: PCT/IB2016/001179

§ 371 Date: Dec. 22, 2017

Related U.S. Application Data
(60) Provisional application No. 62/320,283, filed on Apr. 8, 2016.
(60) Provisional application No. 62/184,924, filed on Jun. 26, 2015.

(51) Int. Cl. A61B 5/16; A61B 5/048; A61B 5/00; G16H 50/20

(52) U.S. Cl. CPC A61B 5/165; A61B 5/048; A61B 5/6803; G16H 50/20; A61B 5/4094; A61B 5/4884; A61B 5/168

(57) ABSTRACT

Computer-implemented systems and methods are provided for determining indications of a mental state of a subject. A computing system may receive at least one electrophysiological signal from at least one pair of electrodes on a head of a subject. The system may store at least one threshold bio-electrical value, and compare a plurality of portions of the at least one electrophysiological signal with the stored value. The system may determine, based on the comparing, identities of portions of the at least one electrophysiological signal that surpass the stored at least one threshold bio-electrical value. The system may also determine the existence of a portion sequence among the portions of the at least one electrophysiological signal that surpass the stored at least one threshold bio-electrical value. And the system may output an indication of a mental state of the subject when it is determined that the portion sequence exists.

### RELATED APPLICATIONS

This application claims the benefit of U.S. Provisional Application No. 62/320,283 filed Apr. 8, 2016, and U.S. Provisional Application No. 62/184,924 filed Jun. 26, 2015. Each patent application identified above is hereby incorporated by reference in its entirety.

### BACKGROUND OF THE INVENTION

EEG is the recording of electrical activity along the scalp. EEG measures voltage fluctuations resulting from ionic current flows within the neurons of the brain. In clinical contexts, EEG refers to (i) the recording of the brain's spontaneous electrical activity over a period of time as recorded from multiple electrodes placed on the scalp; and (ii) the recording of the brain's electrical activity in temporal relation to external events (stimuli or actions)—termed Event Related Potentials (ERP). Diagnostic applications generally focus on the spectral content of EEG, that is, the type of neural oscillations that can be observed in EEG signals, or on specific ERP waveforms.

Medical grade EEG technology has recently been simplified and adapted for use in consumer products. In some aspects, these consumer EEG systems use a relatively small number of dry electrodes, forgoing conductivity gel. These new EEG systems have been implemented in traditional headset bands and paired with EEG analysis and monitoring applications running on mobile and stationary computing devices. But because these new EEG systems typically use small numbers of dry electrodes, they typically produce poor quality raw EEG signals and few sample channels.

Existing EEG analysis and monitoring applications are typically limited by these poor quality raw EEG signals and few sample channels. Accordingly, a need exists for improved systems and methods for processing EEG signals to determine mental state. These improved systems and methods may be used with limited numbers of sensors and reduced sampling frequencies, enabling a range of novel applications.

### SUMMARY

The disclosed computer-implemented systems and method generate indications of the mental state of a subject using received electrophysiological signals. These indications may include markers and indices for use in a range of novel applications. In some aspects, these indications may concern attentiveness, comfort, or focus of a subject.

The disclosed embodiments may include, for example, a method for converting an EEG signal into markers that reflect the attentiveness, comfort or focus of a subject comprises: continuously measuring a subject's EEG during an activity by an electrode assembly to create an EEG sample; and by a computing device in communication with the assembly: filtering the sample to a frequency range of interest; grading the wave data of the filtered sample to create wave values; applying wave value thresholds to the wave values to exclude wave values that fall outside the thresholds, the remaining wave values being crossing values; identifying sequences of the crossing values as well crossing values that are not part of the sequences; and calculating the markers based on the identified sequences.

In some embodiments, measuring a subject's EEG comprises: providing an electrode assembly comprising at least one measuring electrode and one reference electrode; providing a computing device attached to the assembly; attaching the electrode assembly to the subject; and measuring the subject's EEG by the electrode to create an EEG sample and transferring the sample to the device.

In some embodiments, the markers or indices are used in an application from the group consisting of: neuropsychiatric treatment management; training and practice management; neural rehabilitation treatment management; and neuro-marketing and content selection and/or evaluation; training optimization, gaming.

According to further embodiments, a method for converting an Event Related Potential (ERP) into an index that reflects the attention responsiveness of a subject comprises: measuring a subject's ERP using an electrode assembly in response to a repeated stimulus to create an ERP sample; and by a computing device in communication with the assembly: filtering the sample using a low pass filter; grading the wave data of the filtered sample to create wave values; comparing wave values in a period after each of the repeated stimulus to wave values for a similar period before each of the repeated stimulus to determine stimuli responsiveness to each of the stimuli; and calculating the ratio of stimuli responsiveness to the number of the repeated stimuli to determine an attention responsiveness index.

According to further embodiments, a system for converting an EEG signal into markers that reflect the attentiveness, comfort or focus of a subject comprises: an electrode assembly comprising at least one measuring electrode and one reference electrode; wherein the electrode assembly may be attached to the subject for continuously measuring the subject's EEG to create an EEG sample; a computing device attached to the assembly; wherein the EEG sample may be transferred from the assembly to the device; wherein the device may be operative to filter the sample to a frequency range of interest; grade the wave data of the filtered sample to create wave values; apply wave value thresholds to the wave values to exclude wave values that fall outside the thresholds, the remaining wave values being crossing values; identify sequences of the crossing values as well crossing values that are not part of the sequences; and calculate the markers based on the identified sequences.

It is to be understood that both the foregoing general description and the following detailed description are exemplary and explanatory only and are not restrictive of the disclosed embodiments, as claimed.

### BRIEF DESCRIPTION OF THE DRAWINGS

The drawings are not necessarily to scale or exhaustive. Instead, emphasis is generally placed upon illustrating the principles of the subject matter described herein. The accompanying drawings, which are incorporated in and constitute a part of this specification, illustrate several embodiments consistent with the disclosure and together with the description, serve to explain the principles of the disclosure. In the drawings:

FIG. 1 depicts an exemplary electrode assembly.

FIG. 2 depicts an exemplary electrode placement on the scalp of a subject.

FIG. 3 depicts a schematic of an exemplary system for determining mental state using electrophysiological signals.

FIG. 4 depicts a schematic of an exemplary computing systems suitable for determining mental state using electrophysiological signals.

FIG. 5 depicts a flowchart illustrating an exemplary method for determining mental state using electrophysiological signals.

FIGS. 6A and 6B provide an exemplary illustration of signal conditioning.

FIGS. 7A-7D depict exemplary illustrations of four portions of an electrophysiological signal.

FIGS. 8A and 8B depict satisfaction of exemplary amplitude criteria.

FIG. 8C depicts marker extraction on different timescales.

FIGS. 9A and 9B depict generation of exemplary index values.

FIG. 10 depicts an exemplary flowchart showing operations in a method for determining mental state using electrophysiological signals.

FIG. 11 depicts an exemplary illustration of frequency components of an event-related potential.

FIGS. 12A and 12B depict an exemplary determination of a responsiveness value using an event related potential.

FIGS. 13A and 13B depict an exemplary determination of a responsiveness index using responsiveness values.

FIG. 14 depicts an example of tracking of indices over time.

FIG. 15 depicts an exemplary flowchart showing operations in a method for using mental state to improve performance.

FIGS. 16A and 16B depict measured mental states during rehabilitation therapy.

FIGS. 17A-17H concern a study of brain engagement feedback during rehabilitation therapy.

FIG. 18 depicts a schematic of an exemplary system for mobile mental state tracking.

FIGS. 19A and 19B depict examples of mental state tracking for migraine detection.

FIG. 20A depicts a schematic of an exemplary system for mental state tracking using sensor information.

FIGS. 20B-20F concern responsiveness as a predictor of mental and physiological state.

FIG. 20G depicts an example of using BEI for evaluation and prediction of responsiveness to a pharmacological treatment for depression.

FIG. 21 depicts a schematic of an exemplary system for providing recommendations using a mental state database.

FIGS. 22-24 depict an example of measured comfort index and focus events during exposure to different pieces of multimedia content.

FIGS. 25A and 25B depict exemplary comfort index patterns and interpretations.

FIG. 26 illustrates determination of focus events at a ˜1 second resolution during the commercial.

FIGS. 27-29 depict evaluation of user reaction to content.

FIG. 30 depicts detection of comfort index in response to content played for use with a media selection algorithm to automatically select multimedia content.

### DETAILED DESCRIPTION

The disclosed systems and methods may concern generating indications of the mental state of a subject from received electrophysiological data. In some aspects, the electrophysiological data may include at least one of electroencephalogram (EEG), event-related potential (ERP), and electromyogram (EMG) data, or any combination of this data. The indications may concern at least one of engagement, focus, responsiveness, and comfort of the subject. For example, as disclosed below, the indicators may comprise at least one of attentiveness markers, comfort markers, or focus event markers. Indices may be derived from these markers. These indices may include at least one of a brain engagement index (BEI), an attention responsiveness index, and a comfort index. The disclosed systems and methods may also concern applications of these indications to treatment management, computer-based training, and content evaluation.

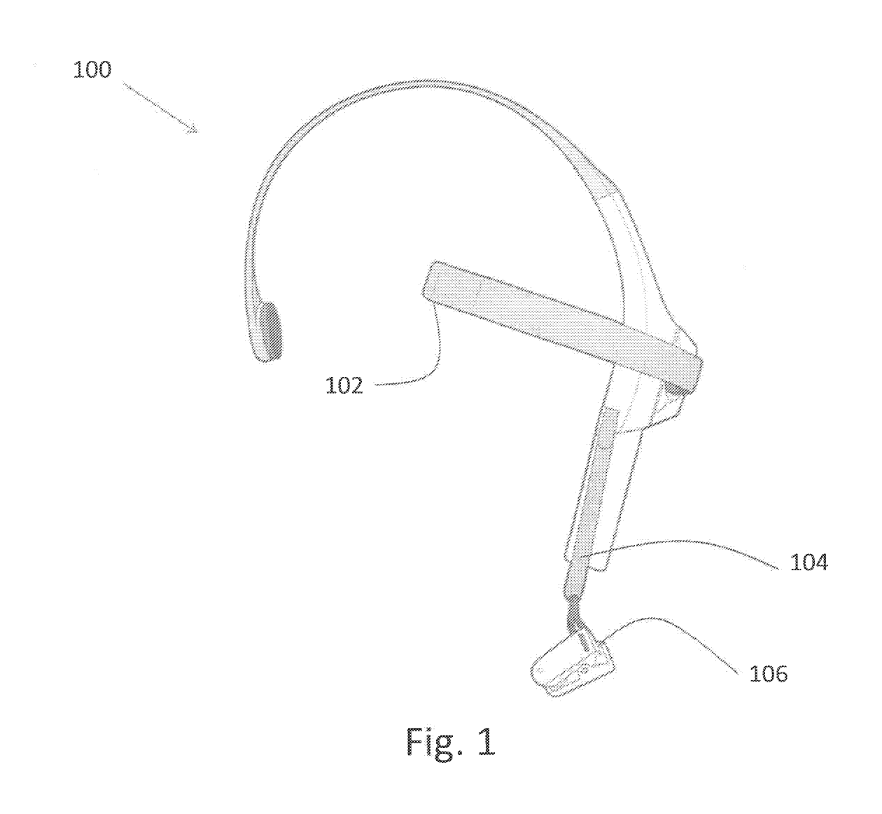

FIG. 1 depicts an exemplary electrode assembly 100 consistent with disclosed embodiments. In some aspects, the exemplary electrode assembly 100 may be positioned on the head of a user. In certain aspects, the exemplary electrode assembly 100 may include one or more pairs of measurement electrodes. For example, exemplary electrode assembly 100 may include a measurement electrode 102 in the central-frontal region. Measurement electrode 102 may be configured to form an electrode measurement pair with a reference electrode, such as reference electrode 104. In some embodiments, reference electrode 104 may serve as a reference electrode for multiple measurement electrode pairs, according to methods known to one of skill in the art. In some embodiments, electrode assembly 100 may comprise a headset, such as a NeuroSky® EEG headset.

Reference electrode 104 may be connected to exemplary electrode assembly 100, consistent with disclosed embodiments. In some aspects, exemplary electrode assembly 100 may be configured to situate reference electrode 104 at a reference point on the head of the subject. In some aspects, exemplary electrode assembly 100 may be configured to attach reference electrode 104 to the skin of the subject on or near the ear of the subject. For example, reference electrode 104 may be attached to the earlobe. Reference electrode 104 may be attached using attachment clip 106. In various aspects, reference electrode 104 may be attached to another body part of the subject.

Measurement electrode 102 may be connected to exemplary electrode assembly 100, consistent with disclosed embodiments. In some aspects, exemplary electrode assembly 100 may be configured to situate reference electrode 104 at a measurement point on the head of the subject. In various aspects, exemplary electrode assembly 100 may be configured to electrically connect measurement electrode 102 to the head of the subject. Measurement electrode 102 may be electrically connected to the subject using at least one of a strap, band, frictional adjustment of a component of exemplary electrode assembly 100 (e.g., an electrode arm of exemplary electrode assembly 100), implantation or partial implantation of measurement electrode 102, suturing, hooking up, and gluing. As would be appreciated by one of skill in the art, other methods of electrically connecting measurement electrode 102 may be used, and the particular method is not intended to be limiting.

Electrode assembly 100 may be configured as a preamplifier, consistent with disclosed embodiments. For example, electrode assembly 100 may be configured to perform at least one of isolation, amplification, and filtering of a voltage difference measured across measurement electrode 102 and reference electrode 104. Electrode assembly 100 may be configured to provide the measured voltage difference between the measurement electrode 102 and reference electrode 104 after amplification. One or more electrodes of electrode assembly 100 may be a dry electrode (i.e. an electrode that does not require conductivity gel).

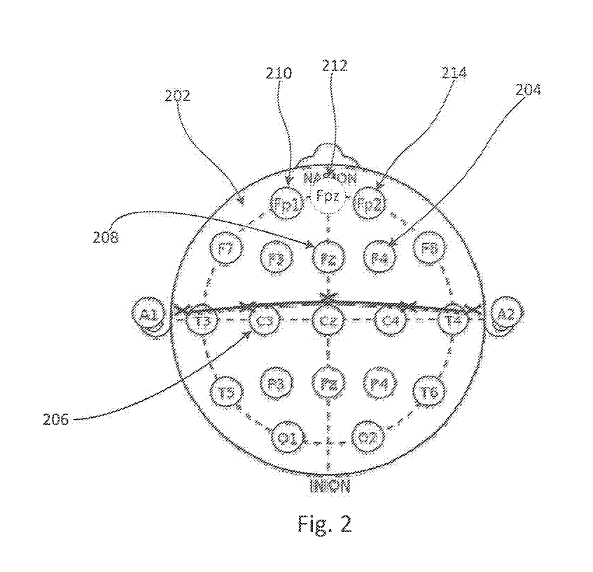

FIG. 2 depicts an exemplary electrode placement on the scalp of a subject consistent with disclosed embodiments. In some aspects, this electrode placement may comprise a 10-20 montage of electrodes on scalp 202 of the subject. As shown in FIG. 2, in certain aspects the electrodes may be placed at positions on scalp 202 including position 204 (F4), position 206 (C3), and a distant reference (not shown). In some aspects, the electrodes may be placed at positions on scalp 202 including position 208 (Fz), position 210 (FP1), position 212 (FPz), position 214 (FP2) and a distant reference (not shown). In some aspects, the position of a measurement electrode, such as measurement electrode 102, may be central, frontal or prefrontal. In certain aspects, the position of a measuring electrode, such as measurement electrode 102, may be near the ear, on a posterior scalp region, or located elsewhere on the scalp. In some embodiments, a measuring electrode (e.g. measurement electrode 102) may be located in the ear of the subject. For example, the measurement electrode may be integrated into earphones.

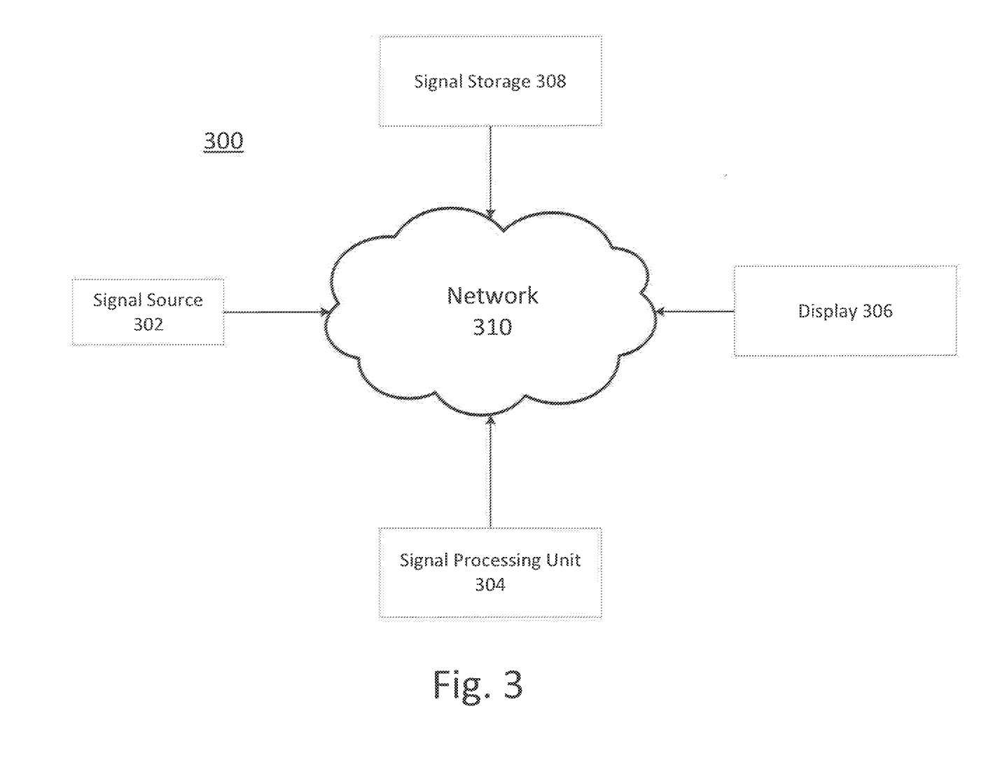

FIG. 3 depicts a schematic of an exemplary system for determining mental state using electrophysiological signals (i.e. mental state system 300) consistent with disclosed embodiments. In some embodiments, mental state system 300 may comprise signal source 302, signal processing unit 304, display 306, and signal storage 308. Consistent with disclosed embodiments, these components may be located on a single device, or may be distributed among one or more devices. In certain embodiments, mental state system 300 may only include a subset of these components. For example, in some embodiments mental state system 300 may not include display 306 or signal storage 308.

Signal source 302 may comprise a source of electrophysiological signals, consistent with disclosed embodiments. In certain embodiments, signal source 302 may comprise a signal gathering device. For example, signal source 203 may comprise electrodes. As an additional example, signal source 203 may comprise at least one of preamplifiers, isolators, filters, and transceivers. In some embodiments, signal source 203 may comprise electrode assembly 100. In certain aspects, signal source 302 may be configured to provide data to one or more of signal processing unit 304, display 306, and signal storage 308. In some embodiments, signal source 302 may comprise a source of processed electrophysiological signals (e.g., conditioned, filtered, digitized, or otherwise processed according to methods known to one of skill in the art). For example, signal source 302 may comprise a network socket. In certain aspects, signal source 302 may be configured to provide processed electrophysiological signals in a data stream. Signal source 302 may be configured to provide data obtained at another geographic location or another time. For example, signal source 302 may be configured to provide data gathered during testing sessions for offline processing at a later time, or at another location.

Signal processing unit 304 may be configured to receive electrophysiological signals, consistent with disclosed embodiments. In some aspects, signal processing unit 304 may be configured to determine mental states by processing the received electrophysiological signals. In some embodiments, signal processing unit 304 may be configured to receive the electrophysiological signals from signal source 302. In various embodiments, signal processing unit 304 may be configured to receive the electrophysiological signals from signal storage 308. In some aspects, signal processing unit 304 may comprise a wearable device (e.g. a smartwatch or fitness tracker), smartphone, tablet, laptop, desktop, workstation, server, or any combination of such devices.

Display 306 may be configured to display information received or generated by mental state system 300, consistent with disclosed embodiments. In some aspects, display 306 may comprise an electronic display. This electronic display may comprise part of a computing device, such as a wearable device display, smartphone display, tablet display, laptop display, or all-in-one system display. This electronic display may comprise a stand-alone display, such as a television or computer monitor. In some embodiments, display 306 and signal processing unit 304 may comprise components of the same computing device. In various embodiments, display 306 and signal processing unit 304 may comprise components of different computing devices.

Signal storage 308 may comprise a non-transitory memory (e.g. hard disk drive, solid state drive, flash memory, magnetic tape, ROM, optical disk, or similar non-transitory memory), consistent with disclosed embodiments. In some embodiments, signal storage 308 may be configured to store electrophysiological signals. In various embodiments, signal storage 308 may be configured to store data resulting from the processing of electrophysiological signals. For example, signal storage 308 may be configured to store at least one of markers and indices generated from electrophysiological signals. In some embodiments, signal storage 308 may be configured to receive electrophysiological signals from signal source 302. In certain embodiments, signal storage 308 may be configured to provide stored electrophysiological signals to signal processing unit 304.

Network 310 may be configured to enable communication between components of mental state system 300, and/or communication between components of mental state system 300 and another system. For example, network 310 may be any type of network (including infrastructure) that provides communications, exchanges information, and/or facilitates the exchange of information between components of mental state system 300. For example, network 310 may be the Internet, a Local Area Network, a serial or parallel port connection (e.g. a USB or firewire connection), computer bus, or other suitable connection(s).

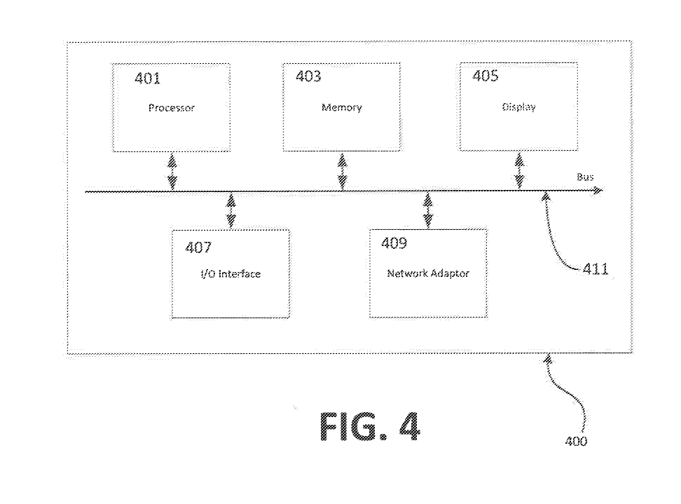

FIG. 4 depicts a schematic of an exemplary computing device 400 suitable for determining mental state using electrophysiological signals, consistent with disclosed embodiments. In some aspects, one or more components of mental state system 300 may be implemented using computing device 400. As a non-limiting example, in some embodiments, at least one of signal source 302, signal processing unit 304, display 306, and signal storage 308 may be implemented using exemplary computing device 400.

According to some embodiments, computing device 400 includes a processor 401, memory 403, display 405, I/O interface(s) 407, and network adapter 409. These units may communicate with each other via bus 411, or wirelessly. The components shown in FIG. 4 may reside in a single device or multiple devices.

In various embodiments, processor 401 may be one or more microprocessors or central processor units performing various methods in accordance to the embodiment. Memory 403 may include one or more non-transitory memories, such as computer hard disks, solid state disks, flash memory, random access memory, removable storage, and remote computer storage. In various embodiments, memory 403 stores various software programs executed by processor 401. For example, memory 403 may be configured to store instructions, such as computer programs, for performing operations consistent with disclosed embodiments. I/O interfaces 407 may include keyboard, a mouse, an audio input device, a touch screen, or an infrared input interface. Network adapter 409 enables computing device 400 to communicate over computer networks, such as network 310. In various embodiments, network adapter 409 may include a wireless wide area network adapter, or a local area network adapter.

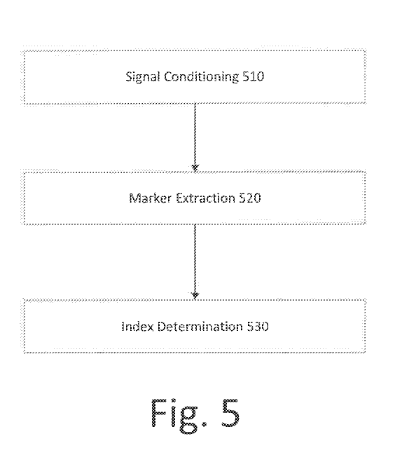

FIG. 5 depicts a flowchart illustrating an exemplary method for determining an index indicative of a mental state. In some embodiments, one or more computing devices, such as exemplary computing device 400, may be configured to perform this exemplary method. In some embodiments, this method may include identifying sequences of maxima and minima in EEG signals. The identified sequences may be used to generate an index of mental state (e.g., the BEI). In some embodiments, this method may include signal conditioning step 510, marker extraction step 520, and index determination step 530.

In signal conditioning step 510, a received electrophysiological signal may be conditioned in preparation for extraction of indications of mental state. In some embodiments, such operations may be performed by one or more computing devices. For example, signal processing unit 304 may be configured to perform signal conditioning in step 510. Step 510 may comprise performing a sequence of operations on received electrophysiological signal prior to extracting indications of mental state. These operations may include at least one of receiving, isolating, amplifying, filtering, and performing analog to digital conversion of an electrophysiological signal.

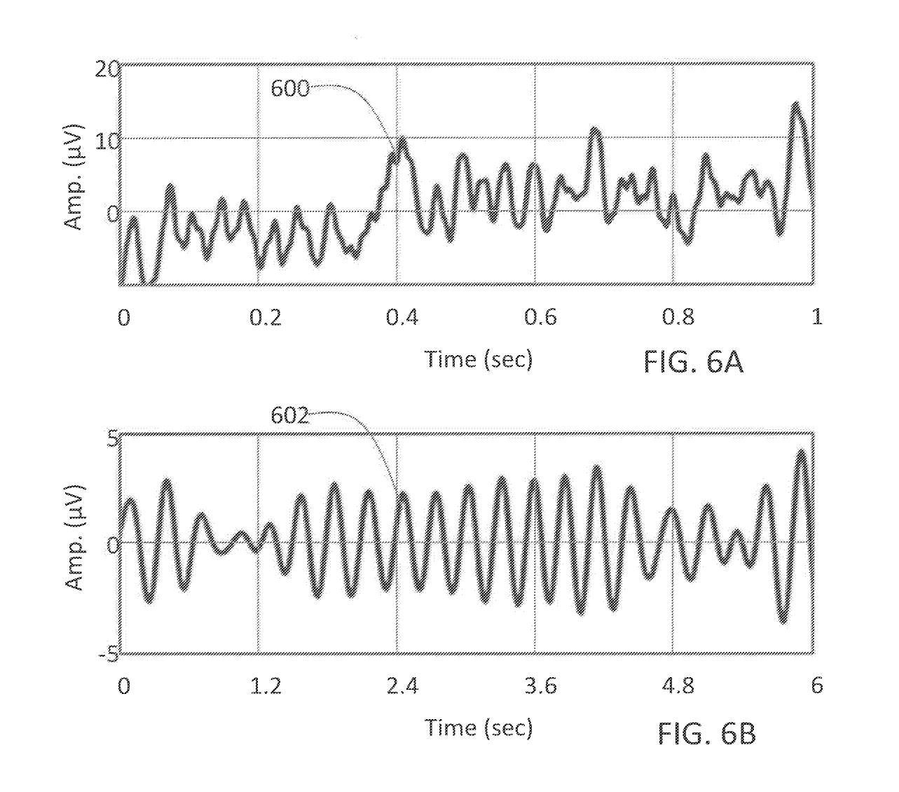

As a non-limiting example, signal source 302 may comprise at least one pair of electrodes on a head of a subject, and step 510 may comprise receiving at least one electrophysiological signal from signal source 302. Signal conditioning may comprise filtering the received at least one electrophysiological signal, consistent with disclosed embodiments. For example, signal processing unit 304 may be configured to receive unfiltered signals, such as the signals shown in FIG. 6A. In some aspects, filtering may be accomplished using one or more filters. The one or more filters may be configured to attenuate electrophysiological signal components outside a EEG frequency band recognized by one of skill in the art. For example, the one or more filters may be configured to attenuate signal components outside at least one of the delta and theta EEG frequency bands. For example, the one or more filters may be configured to attenuate signal components with frequencies below 1 Hz. In another example, the one or more filters may be configured to attenuate signal components with frequencies above 4 Hz. As a further example, the one or more filters may be configured to attenuate signal components with frequencies above 8 Hz. In some embodiments, the one or more filters may include at least one of a low-pass filter, band-pass filter, and high-pass filter. The resulting filtered signal may resemble the filtered signal shown in FIG. 6B.

In marker extraction step 520, system 300 may be configured to extract markers from one or more portions of the electrophysiological signal. In some embodiments, step 520 may be performed on portions of the received electrophysiological signal processed in step 510. In some aspects, signal processing unit 304 may be configured to extract the markers. In various aspects, the markers may be extracted repeatedly. For example, signal processing unit 304 may be configured to extract markers from successive portions of the electrophysiological signal. In some aspects, the portions may be overlapping. As a non-limiting example, signal processing unit 304 may be configured to extract a first marker from a first portion of an electrophysiological signal ranging from t+0 seconds to t+120 seconds, and the next marker from a second portion of an electrophysiological signal ranging from t+60 seconds to t+180 seconds. In various aspects, the portions may be contiguous. As a non-limiting example, signal processing unit 304 may be configured to extract a first marker from a first portion of an electrophysiological signal ranging from t+0 seconds to t+120 seconds, and the next marker from a second portion of an electrophysiological signal ranging from t+120 seconds to t+240 seconds. In some aspects, the portions may be non-contiguous. As a non-limiting example, signal processing unit 304 may be configured to extract a first marker from a first portion of an electrophysiological signal ranging from t+0 seconds to t+120 seconds, and the next marker from a second portion of an electrophysiological signal ranging from t+240 seconds to t+360 seconds.

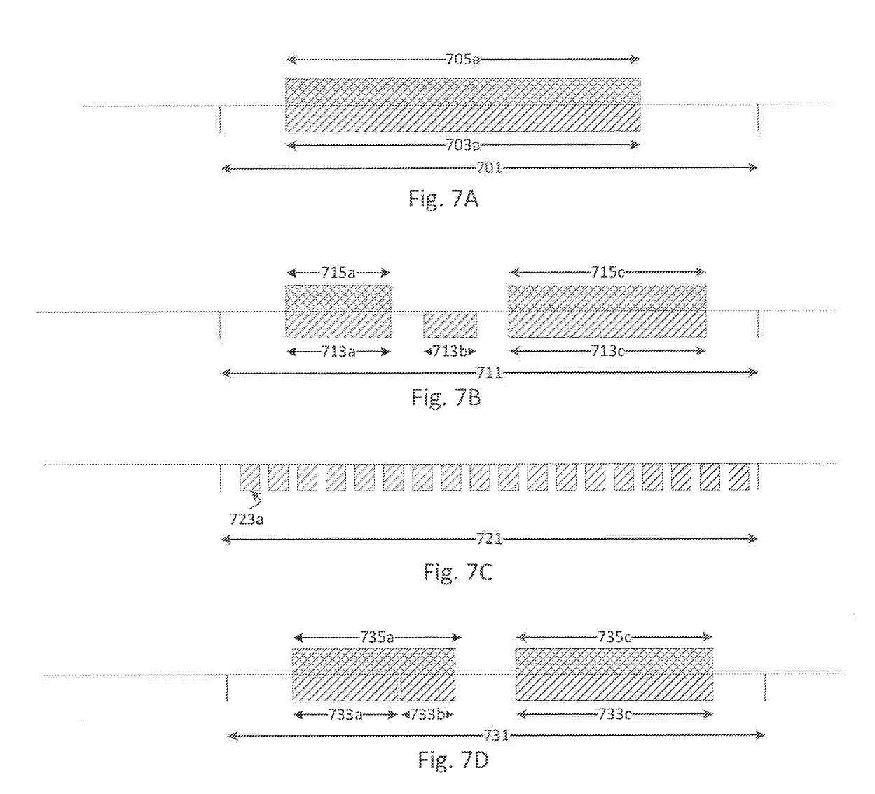

FIGS. 7A-7D depict exemplary illustrations of four portions of such a processed electrophysiological signal (i.e. portions 701-731). In some embodiments, these portions may comprise time intervals of the electrophysiological signal. In some aspects, a time interval may have a duration, a beginning time and an ending time. As a non-limiting example, a portion may comprise a preceding time interval. The beginning time may be between 300 seconds and 100 milliseconds before the ending time. In some aspects, the ending time may be the current time, or within 50 milliseconds of the current time.

Marker extraction step 520 may comprise determining first sub-portions of the portions that satisfy criteria, consistent with disclosed embodiments. The criteria may include amplitude criteria and duration criteria. In some embodiments, marker extraction step 520 may comprise determining at least one segment that satisfies a first criteria. This first criteria may be an amplitude criteria. In some aspects, marker extraction may comprise determining this amplitude criteria. The amplitude criteria may be determined based on at least one of the amplitude of the electrophysiological signal, and the amplitude of the portion of the electrophysiological signal.

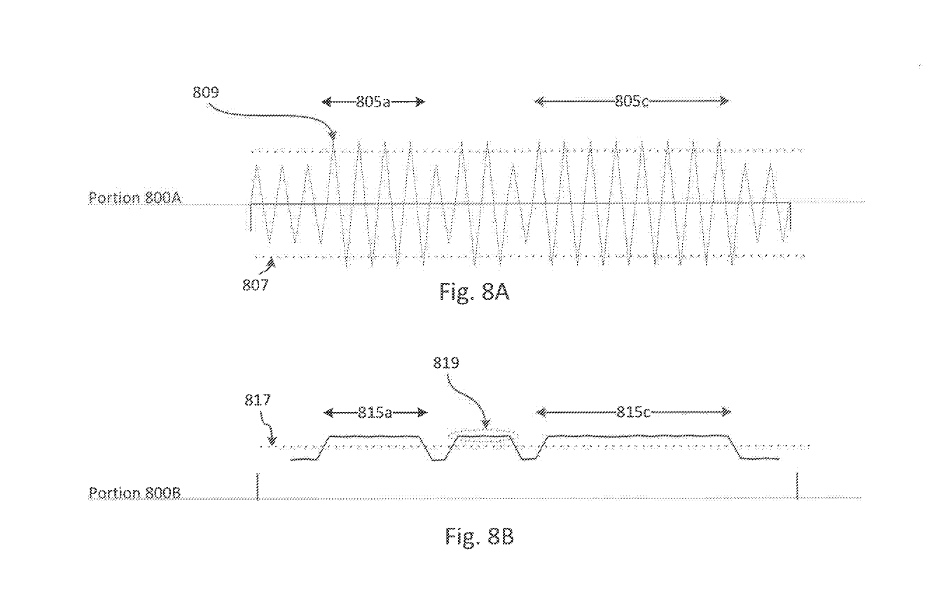

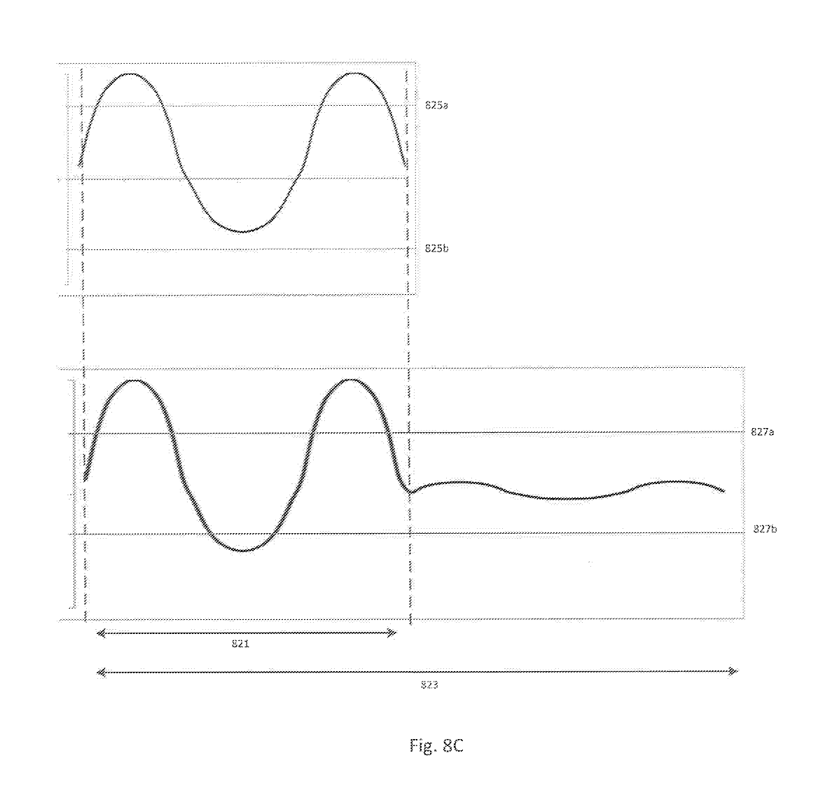

As a non-limiting example, as depicted in FIGS. 8A and 8B, segments of the electrophysiological signal may satisfy the amplitude criteria. In some embodiments, segments of the electrophysiological signal surpassing an amplitude threshold may satisfy the amplitude criteria. In some embodiments, the amplitude criteria may depend on a statistical measure of values of the electrophysiological signal, such as percentiles, averages, and standard deviations. For example, the amplitude threshold may be the top 20% or the top 40% of electrophysiological signal values. As an additional example, the amplitude threshold may be the average plus and minus a number of standard deviations. In some aspects, the amplitude threshold may be computed over at least part of the electrophysiological signal. For example, the amplitude threshold may be computed over an entire portion of the electrophysiological signal, such as portion 800a or 800b. As an additional example, the amplitude threshold may be computed over the entire electrophysiological signal.

In some embodiments, the amplitude threshold may be predetermined. In various aspects marker extraction step 520 may comprise storing the determined amplitude threshold in a memory, such as a non-transitory memory of signal processing unit 304 or signal storage 308. In certain aspects, marker extraction step 520 may comprise retrieving this amplitude threshold from a memory, such as a non-transitory memory of signal processing unit 304 or signal storage 308.

As depicted in FIG. 8A, the amplitude criteria may be satisfied by segments of the electrophysiological signal. In some aspects, such segments may comprise a sequence of half-waves. Such segments may have peaks (e.g. peak 809) with amplitudes surpassing the amplitude threshold. For example, portion 800A may comprise a sequence of peaks surpassing the amplitude threshold (e.g., segments 805a and 805c). FIG. 8A depicts an exemplary segment 805a comprising four positive-going half waves with peaks having amplitudes greater than amplitude threshold 807 and four negative-going half waves with peaks having amplitudes greater than amplitude threshold 807.

As depicted in FIG. 8B, the amplitude criteria may be satisfied by a function of the electrophysiological signal. As a non-limiting example, FIG. 8B depicts the envelope of the electrophysiological signal depicted in FIG. 8A. In some aspects, the amplitude criteria may be satisfied when segments of the envelope (e.g. segments 815a and 815c) surpass amplitude threshold 817. For example, extreme values (e.g. extreme values 819) may exceed the amplitude threshold 817. Additional examples of functions of the electrophysiological signal consistent with disclosed embodiments include rectified, filtered, and/or integrated versions of the electrophysiological signal. As an additional example, the function of the electrophysiological signal may depend on the half-wave integral, full-wave integral, multiwave integral, wave amplitude, or wave amplitude deviation between consecutive half-waves or full waves, wave area, or a combination of these features, of the electrophysiological signal; or any other wave manipulation or wave discretization technique.

In some embodiments, marker extraction step 520 may comprise determining second sub-portions that satisfy both the first criteria and a second criteria (e.g. sub-portion 705a and sub-portions 715a and 715c). The second criteria may comprise a durational criteria. In some aspects, this durational criteria may be satisfied when the second sub-portions exceed the first criteria for at least a first duration. In some aspects, two or more segments of an electrophysiological signal satisfying the first criteria may collectively satisfy the second criteria. For example, such two or more segments may each be separated by less than a minimum time. As an additional example, such two or more segments may collectively have greater than a second duration. In some aspects, the second duration may be greater than the first duration.

As depicted in FIG. 8C, marker extraction may occur on differing timescales, consistent with disclosed embodiments. System 300 may be configured to analyze the received electrophysiological signal on a first timescale, analyzing a first portion 821 for the presence of a first marker. System 300 may also be configured to analyze the received electrophysiological signal on a second timescale, analyzing a second portion 823 for the presence of a second marker. In some embodiments, as shown in FIG. 8C, the first portion and the second portion may be partially overlapping. As would be appreciated by one of skill in the art, differences between first portion 821 and second portion 823 may affect whether system 300 identifies a marker within each of these segments. For example, as described above with regards to FIG. 8B, in some embodiments system 300 may be configured to determine whether segments of the electrophysiological signal satisfy an amplitude criteria. In some embodiments, the amplitude criteria for a portion may depend on the values of the electrophysiological signal within the portion. For example, as shown in FIG. 8C, the amplitude criteria 825a and 825b are greater than the amplitude criteria 827a and 827b because, in this non-limiting example, the average value of the electrophysiological signal over the shorter first portion 821 may be greater than the average value of the electrophysiological signal over the longer second portion 823. Consequently, three half-waves satisfy the amplitude criteria for second portion 823, while only two half-waves satisfy the amplitude criteria for first portion 821. System 300 may be configured to generate a marker corresponding to the three half-waves satisfying the amplitude criteria for second portion 823, but not the two half-waves satisfying the amplitude criteria for first portion 821. System 300 may therefore be configured to analyze the received electrophysiological signal over multiple timescales to determine markers apparent at these differing timescales.

FIGS. 7A-7D depict exemplary determinations of first sub-portions and second sub-portions, consistent with disclosed embodiments. In FIG. 7A, a single continuous segment of portion 701 satisfies the first criteria. A first sub-portion 703a may correspond to this segment. As this segment also satisfies the second criteria, a second sub-portion 705a may also correspond to this segment. In FIG. 7B, three segments of portion 711 satisfy the first criteria. Three first sub-portions may correspond to these segments (i.e. sub-portions 713a-713c). But only the first and third segments satisfy the second criteria. Two second sub-portions may correspond to these first and third segments (i.e. sub-portions 715a and 715c). No second sub-portion corresponds to the second segment. In FIG. 7C, eighteen segments of portion 721 satisfy the first criteria. Eighteen first sub-portions may correspond to these segments (e.g. sub-portion 723a). But none of the eighteen segments satisfy the second criteria, so no second sub-portions correspond to any of the eighteen segments. In FIG. 7D, three segments satisfy the first criteria. Three first sub-portions may correspond to these three segments (i.e. sub-portions 733a-733c). A second sub-portion 735a may correspond to the first two of these three segments, as they are separated by less than a minimum time and collectively exceed the second duration. A second sub-portion 735c may correspond to the third of these three segments, as this third segment exceeds the first duration. In some aspects, the second duration may exceed the first duration. In various aspects, the first and second duration may be equal.

In certain aspects, each of the first and second durations may be at least a few hundred milliseconds. In some aspects, these durations may be between 100 and 1000 milliseconds. In various aspects, these durations may be between 50 and 200 milliseconds. In some aspects, these durations may be between 50 and 500 milliseconds. In various aspects, these durations may be between 50 and 2000 milliseconds. In some aspects, these durations may exceed 2000 milliseconds.

Consistent with disclosed embodiments, marker extraction step 520 may comprise determining values based on the determined portions and/or sub-portions. In some embodiments, signal processing unit 304 may be configured to determine the values. Marker extraction step 520 may comprise creating an event marker for one or more second sub-portions. Event markers may comprise data stored in a memory, such as a non-transitory memory of signal processing unit 304 or signal storage 308.

Consistent with disclosed embodiments, event markers may include focus event markers. Focus event markers may indicate short executive function events. Focus event markers may be associated with second sub-portions with durations exceeding a threshold between 50 and 2000 milliseconds. In certain aspects, focus event markers may be associated with second sub-portions with durations exceeding 100 milliseconds. A focus event marker may include a time of the corresponding second sub-portion. This time may be a start time or end time of the corresponding second sub-portion, or a time between the start time and end time of the corresponding second sub-portion.

Consistent with disclosed embodiments, event markers may include attentiveness markers. Attentiveness markers may indicate a degree of attention of the subject. In some aspects, attentiveness markers may depend on a first value of the first sub-portions and a second value of the second sub-portions. For example, attentiveness markers may depend on the second value divided by the first value. This division may yield an attentiveness marker value between zero and one. The attentiveness marker value may vary continuously with either the second value or the first value. For example, the attentiveness marker value may be monotonically nondecreasing with increases in the second value. Similarly, the attentiveness marker value may be monotonically nonincreasing with increases in the first value.

The first value of the first sub-portions may depend on at least one of a total duration of the first sub-portions or the values of the electrophysiological signal within the first sub-portions. For example, where the total duration of the first sub-portions is 100 milliseconds, the first value may be 100 milliseconds. As an additional example, the first value of the first sub-portions may depend on a total number of peaks of the electrophysiological signal within the first sub-portions, a total number of extreme values of the electrophysiological signal within the first portions, or a first function of the values of the electrophysiological signal. Such a first function may be based on half-wave integral, full-wave integral, multiwave integral, wave amplitude, or wave amplitude deviation between consecutive half-waves or full waves, wave area, or a combination of these, or any other wave manipulation or wave discretization technique.

The second value of the second sub-portions may depend on at least one of a total duration of the second sub-portions or the values of the electrophysiological signal within the second sub-portions. For example, where the total duration of the second sub-portions is 100 milliseconds, the second value may be 100 milliseconds. As an additional example, the second value of the second sub-portions may depend on a total number of peaks and/or extreme values of the electrophysiological signal within the second sub-portions, or a second function of the values of the electrophysiological signal. Such a second function may be based on half-wave integral, full-wave integral, multiwave integral, wave amplitude, or wave amplitude deviation between consecutive half-waves or full waves, wave area, or a combination of these, or any other wave manipulation or wave discretization technique. In some aspects, the first function and the second function may be the same function.

Consistent with disclosed embodiments, event markers may include comfort markers. Consistent with drive reduction theory, comfort markers may indicate a degree of relaxation or satisfaction of the subject. In some aspects, comfort markers may depend on a first value of the first sub-portions and a second value of the second sub-portions. For example, comfort markers may depend on the second value divided by the first value. For example, the comfort marker value may be one minus the second value divided by the first value. As described above with respect to attentiveness markers, the first value may depend on at least one of a total duration of the first sub-portions or the values of the electrophysiological signal within the first sub-portions, and the second value may depend on at least one of a total duration of the second sub-portions or the values of the electrophysiological signal within the second sub-portions. The attentiveness marker value may vary continuously with either the second value or the first value. For example, the comfort marker value may be monotonically nonincreasing with increases in the second value. Similarly, the attentiveness marker value may be monotonically nondecreasing with increases in the first value.

The exemplary method of FIG. 5 may additionally include removal of portions of the received electrophysiological signals satisfying a noise criteria. In some aspects, this removal may be performed prior to step 520. For example, this removal may be performed prior to digitization of the received electrophysiological signals. In other examples, this removal may be performed after digitization of the received electrophysiological signals. In some aspects, the noise criteria may comprise an amplitude criteria. For example, an amplitude threshold may be used to distinguish EEG signals from EMG signals, or EEG and EMG signals from 60 Hz noise, according to methods known to one of skill in the art. Portions of the received electrophysiological signals satisfying the amplitude criteria may be removed. In various aspects, the noise criteria may comprise a timing criteria. For example, when electrical stimuli are provided, portions of the received electrophysiological signals within a certain duration of the provided stimuli may be removed. Removal may include deletion of the portion from the received signal, or otherwise preventing use of the portion satisfying the noise criteria during marker extraction step 520.

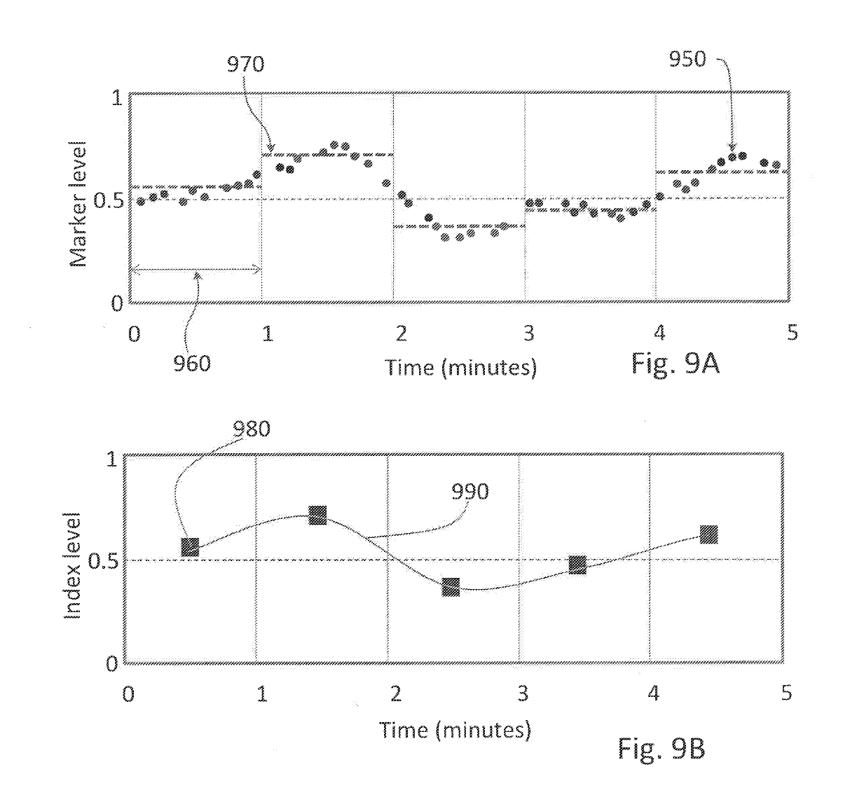

Index determination step 530 may comprise determining an index value from one or more of extracted makers. In some embodiments, index determination step 530 may be performed by signal processing unit 304. As depicted in FIG. 9A, a sequence of marker values (e.g. sequence 950) may be divided into intervals (e.g. interval 960), consistent with disclosed embodiments. In some aspects, an index value (e.g. index value 970) may be computed for each interval. In some embodiments, the duration of the interval may depend on at least one of a type of the marker (e.g., attention marker, comfort marker, etc.), and an application of the index (e.g., evaluating advertising, predicting migraines, etc.). In various aspects, the index value for an interval may be a function of the marker values within the interval. For example, the index value may be a statistic of the marker values, such as a minimum, average, median, variance, or maximum marker value. The index value may also be a combination of such statistics. As depicted in FIG. 9B, a sequence of index values may be constructed (e.g. index sequence 990). In certain aspects, index sequence 990 may include at least one of the index values calculated for the intervals (e.g. index value 980). In some aspects, a brain engagement index may comprise an index generated from attention markers. In various aspects, a comfort index may comprise an index generated from comfort markers.

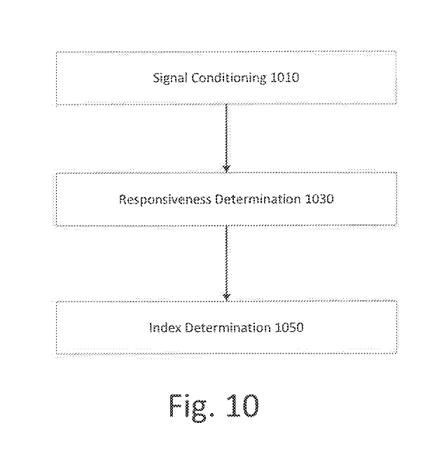

FIG. 10 depicts a flowchart illustrating an exemplary method for determining a responsiveness index indicative of a mental state. In some embodiments, this method may include identifying event-related potentials in an EEG recording. In some embodiments, this method may include signal conditioning step 1010, responsiveness determination step 1030, and responsiveness index determination step 1050.

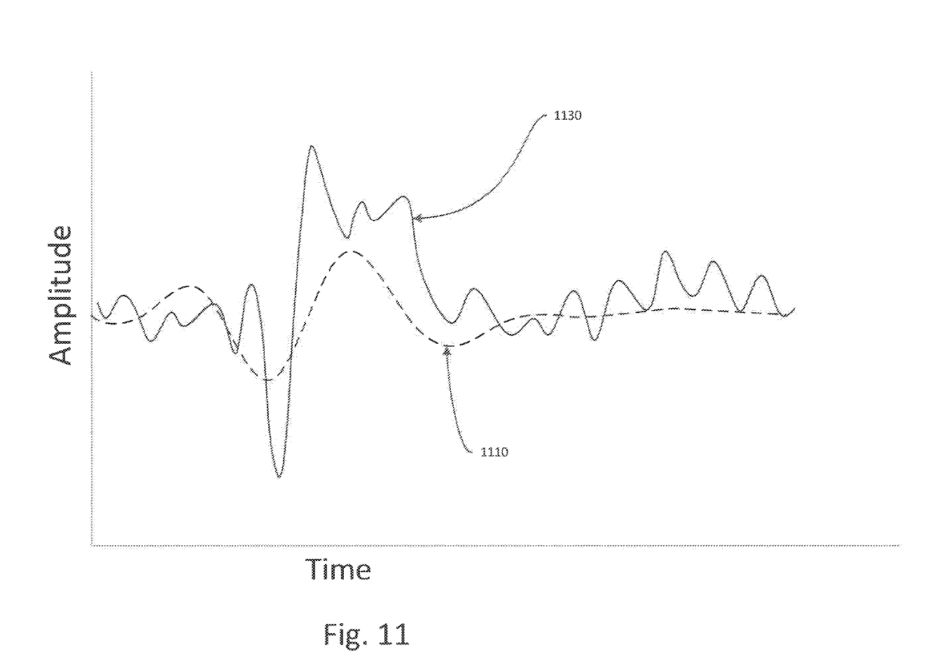

Consistent with disclosed embodiments, signal conditioning step 1010 may resemble signal processing step 510, disclosed above. As depicted in FIG. 11, as a non-limiting example, a received electrophysiological signal, such as signal 1130, may be processed to yield a signal associated with a particular EEG band, such as signal 1110. In some embodiments, this processing may be performed by signal processing unit 304. For example, signal processing unit 304 may be configured to filter signal 1130 with a delta bandpass filter to yield signal 1110. In some aspects, exemplary system 300 may be configured to reject specific types of EMG activity based on the amplitude of the signal. In some aspects, exemplary system 300 may be configured to discard without processing portions of the signal contaminated by specific types of EMG signals.

Responsiveness determination step 1030 may comprise evaluation of the magnitude of the detected signal before and after one or more stimuli, consistent with disclosed embodiments. Potential stimuli consistent with disclosed embodiments encompass sensory stimuli such as auditory, visual, tactile, olfactory, or gustatory stimuli. In some embodiments, the one or more stimuli may comprise auditory oddball stimuli.

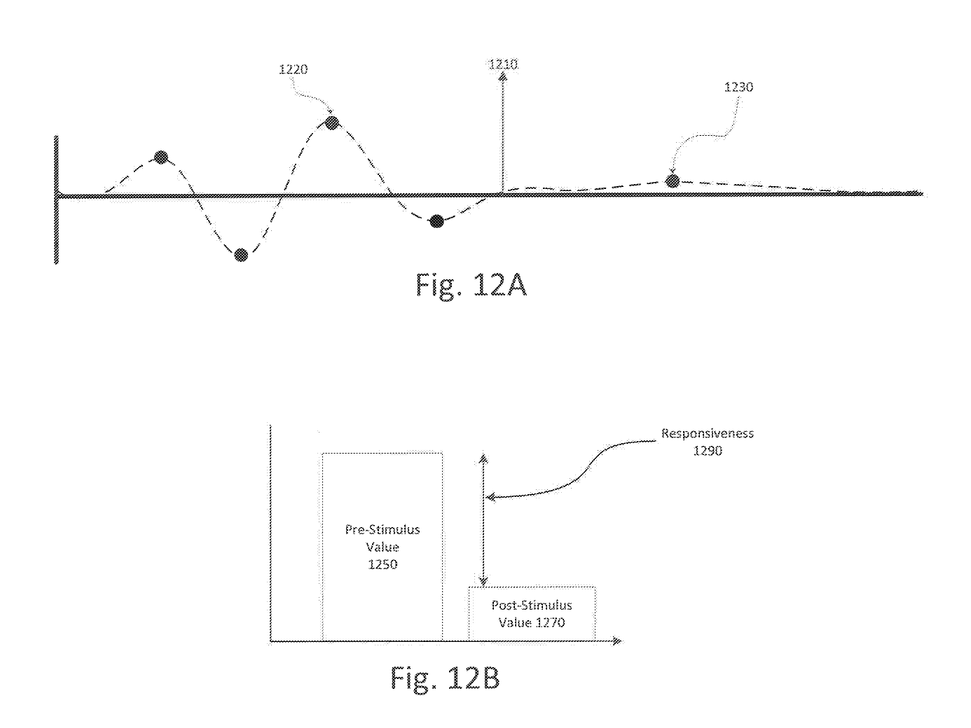

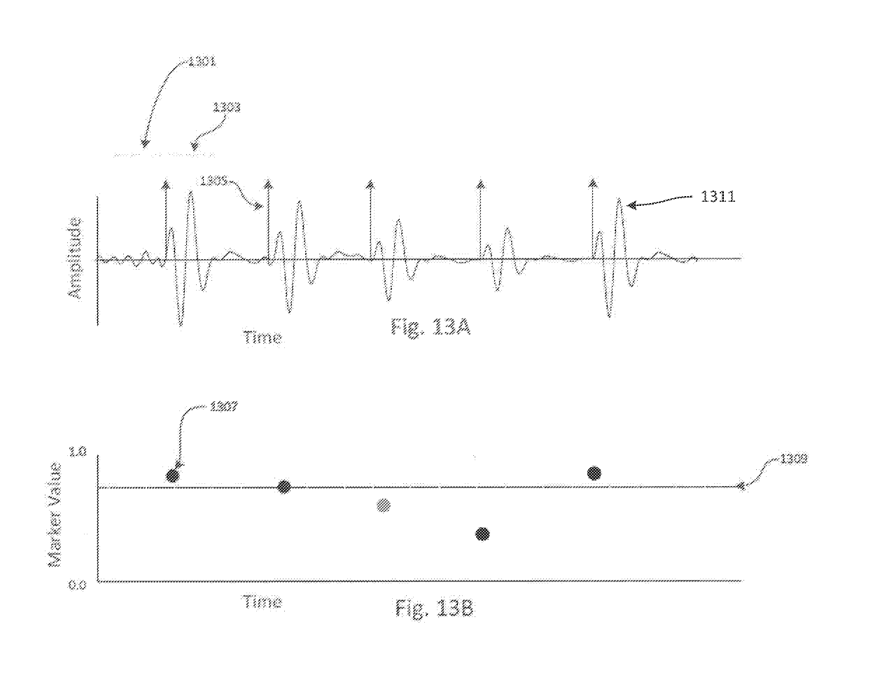

FIGS. 12A and 12B depict an exemplary responsiveness determination, consistent with disclosed embodiments. In some embodiments, signal processing unit 304 may be configured to perform this responsiveness determination. In some aspects, signal processing unit 304 may be configured to determine pre-stimulus value 1250 and post-stimulus value 1270 based on signal 1110.

For example, as shown in FIG. 12A, signal processing unit 304 may be configured to determine the amplitude of peaks prior to the occurrence of a stimulus (e.g. peak 1220) and peaks following the occurrence of a stimulus (e.g. peak 1230). In some embodiments, signal processing unit 304 may be configured to determine pre-stimulus value 1250 based on the values of the pre-stimulus peaks, and/or post-stimulus value 1270 based on the values of the post-stimulus peaks. In various embodiments, signal processing unit 304 may be configured to determine pre-stimulus value 1250 and post-stimulus value 1270 as statistics of the pre-stimulus and post-stimulus peak values, such as minimums, means, medians, or maximums; or other functions of the pre-stimulus and post-stimulus peak values.

As an additional example, signal processing unit 304 may be configured to determine the magnitude of a principal component corresponding to an expected event-related potential prior to the occurrence of a stimulus, and following the occurrence of a stimulus. Signal processing unit 304 may be configured to determine pre-stimulus value 1250 based on the magnitude of the pre-stimulus principal component, and/or determine post-stimulus value 1270 based on the magnitude of the post-stimulus principal component.

Signal processing unit 304 may be configured to determine responsiveness value 1290 as a difference between pre-stimulus value 1250 and post-stimulus value 1270, consistent with disclosed embodiments. In some aspects, this responsiveness value may be normalized by the larger of the pre-stimulus value 1250 and post-stimulus value 1270 to yield a value between zero and one.

System 300 may be configured to determine a responsiveness index in responsiveness index determination step 1050, consistent with disclosed embodiments. In some embodiments, responsiveness index determination step 1050 may be performed by signal processing unit 304. FIGS. 13A and 13B depict the calculation of responsiveness index values from an electrophysiological signal, such as electrophysiological signal 1311. As depicted in FIG. 13A, multiple stimuli (e.g. stimuli 1305), may be presented to the subject. As described above with respect to FIG. 12, pre-stimulus values and post-stimulus values may be generated for time periods before and after each stimulus (e.g. pre-stimulus time period 1301 and post-stimulus time period 1303). As depicted in FIG. 13B, signal processing unit 304 may be configured to determine responsiveness values (e.g. responsiveness value 1307) corresponding to the stimuli. Signal processing unit 304 may be configured to determine a responsiveness index (e.g. responsiveness index 1309) from the responsiveness values. In some aspects, the responsiveness index may be a statistic of these responsiveness values, such as a minimum, mean, median, variance or maximum; a combination of such statistics; or another function of the pre-stimulus and post-stimulus peak values. For example, signal processing unit 304 may be configured to determine intermediate values from sets of responsiveness values, and then determine the responsiveness index from these intermediate values. In some aspects, the intermediate values may comprise a statistic of the responsiveness values for an interval of the electrophysiological signal, such as a minimum, mean, median, or maximum; or another function of these responsiveness values. In various aspects, the responsiveness index may be a statistic of the intermediate values, such as a minimum, mean, median, variance, or maximum; a combination of such statistics; or another function of the intermediate values. For example, system 300 may be configured to determine normalized responsiveness values based on peak values before and after each stimuli in a set of stimuli. System 300 may be configured to determine first intermediate values as the median of these normalized responsiveness values. System 300 may be configured to determine the responsiveness index as the median of intermediate values determined for multiple such sets of stimuli.

System 300 may be configured to generate a responsiveness index using the brain engagement index described with regard to FIGS. 9A and 9B, consistent with disclosed embodiments. In some embodiments, signal processing unit 304 may be configured to determine the brain engagement index for intervals of the received electrophysiological signal between 5 and 15 seconds. In some aspects, the intervals may be between 8 and 12 seconds, or approximately 10 seconds. Signal processing unit 304 may be configured to discard brain engagement index values for invalid intervals, such as those corrupted by noise. For example, signal processing unit 304 may be configured to discard brain engagement index values for intervals corrupted by excessive EMG signals, or 60 Hz noise, or other noise sources recognized by one of skill in the art. Signal processing unit 304 may be configured to calculate the responsiveness index as statistic of the valid brain engagement index values, such as a minimum, mean, median, variance or maximum; a combination of such statistics; or another function of these valid brain engagement index values. In some embodiments, the responsiveness index may be calculated as the median of the three earliest valid periods. Thus system 300 may be configured to determine the responsiveness index from the received electrophysiological signal without regard to the stimuli.

In certain embodiments, signal processing unit 304 may be configured to disregard additional intervals. For example, in certain aspects, signal processing unit 304 may be configured to calculate the responsiveness index from the first three consecutive intervals for which the brain engagement index value differs by less than 0.5, disregarding the remaining samples. In some embodiments, signal processing unit 304 may be configured to generate a responsiveness index based on both responsiveness values and the brain engagement index.

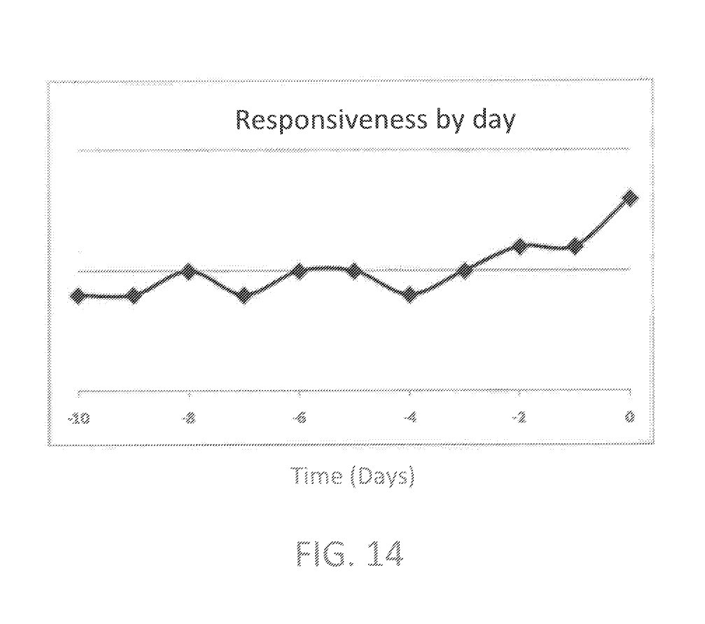

As shown in FIG. 14, system 300 may be configured to store sequences of at least one of a marker, brain engagement index, and responsiveness index. For example, system 300 may be configured to repeatedly determine a value of the marker, brain engagement index, and/or responsiveness index. System 300 may be configured to repeatedly determine such values at regular and/or irregular intervals. For example, system 300 may be configured to determine such values hourly, daily, weekly, monthly, or some combination of these intervals. In some embodiments, the inter-value interval may depend on the most recently measured value, or a difference between the most recently determined value and one or more previously determined values. For example, a large change in values may prompt another measurement at a reduced inter-value interval. In some aspects, system 300 may be configured to store these values as data in a non-transitory memory, for example a non-transitory computer memory associated with one or more of signal storage 308 and signal processing unit 304. System 300 may therefore, in some embodiments, be configured to track at least one of a marker, brain engagement index, and responsiveness index over time. As described below, system 300 may be configured to determine mental states based on these tracked values.

### Exemplary Method for Determining Mental State

According to some embodiments, a system for determining indication of a mental state is disclosed. The system may comprise a computer-readable medium storing instructions and a processor. In some embodiments, when executed by the processor, the instructions cause the processor to perform operations. The operations may include receiving at least one electrophysiological signal from at least one pair of electrodes on a head of a subject. The operations may also include storing at least one threshold bio-electrical value. The operations may further include comparing a plurality of portions of the at least one electrophysiological signal with the stored at least one threshold bio-electrical value. The operations may additionally include determining, based on the comparing, identities of portions of the at least one electrophysiological signal that surpass the at least one threshold bio-electrical value. The operations may further include determining whether a sequence exists among the portions of the at least one electrophysiological signal that surpass the at least one threshold bio-electrical value. And the operations may include outputting an indication of a mental state of the subject when the system determines that the sequence exists.

The at least one pair of electrodes may be only one pair of electrodes. The operations may further comprise, prior to storing, determining the at least one threshold bio-electrical value by analyzing the electrophysiological signal and identifying extremes in the electrophysiological signal. Determining whether the sequence exists may include determining an existence of at least three contiguous extrema exceeding the at least one threshold bio-electrical value. The indication of the mental state may indicate one or more of an attentiveness, focus, comfort, and responsiveness level of the subject. The at least one received electrophysiological signal may include a first electrophysiological signal and a second electrophysiological signal. The operations may further comprise determining a first sequence in the first electrophysiological signal and a second sequence in the second signal and calculating a first measure of mental state based on the first sequence and a second measure of mental state based on the second sequence. An indication of the mental state of the subject may be based on the first measure and the second measure. The first electrophysiological signal may occur in an absence of an artificial stimulus, and the second electrophysiological signal may occur, at least in part, in a presence of the artificial stimulus. Determining the at least one first threshold bio-electrical value may include analyzing the first electrophysiological signal and identifying extremes in the first electrophysiological signal. The stimulus may be selected to assist in predicting an onset of a migraine, and the indication of the mental condition may involve a prediction of migraine onset. The mental condition may relate to at least one of migraine, anxiety, ADD, ADHD, depression, epilepsy and dementia. The operations may further comprise updating a sequence of values based on the first measure and the second measure, and wherein the indication of the mental condition may be based on the sequence of values. The determination of mental condition may be based on the sequence of values and at least one of historical subject population data and historical subject data.

### Neural Rehabilitation Treatment Management

Neural rehabilitation of a patient, for example after a cerebrovascular accident, may benefit from increased patient engagement. Enhanced patient engagement may increase activation of brain regions targeted for rehabilitation (e.g. motor regions for motor rehabilitation). This increased activation may enable formation of compensatory connections. Patient engagement during a rehabilitation session may depend on multiple factors, including the appropriateness of the rehabilitation exercise, given the patient's functional level and mental state (e.g., boredom, exhaustion). Providing appropriate rehabilitation exercises may therefore improve rehabilitation effectiveness.

As would be appreciated by one of skill in the art, neural rehabilitation management may include, for a subject: evaluating neurophysiological state; continuous evaluation of rehabilitation training effectiveness and/or performance of activities of daily living (ADLs); automatic, real-time recommendations and instructions concerning rehabilitation training protocols and/or ADLs; and evaluation of other therapeutic interventions (e.g., drug and/or therapy-based interventions). In some aspects, the subject may have one or more physical or neurological deformities, diseases, or injuries. For example, a subject may have at least one neurological disorder such as a stroke; a movement disorder; impaired limb function; physical injury; traumatic brain injury, cerebral palsy, multiple sclerosis, spinal cord injury, neurodegenerative disease, peripheral nerve disease, and cognitive impairment. Alternatively, the subject may be healthy.

The term “neural rehabilitation” as used herein should not be considered limiting, and may include any behavioral treatment for any neuropsychiatric disorder. Beyond the above mentioned injuries and dysfunctions this also includes behavioral treatment for various psychopathologies (e.g. in the depressive spectrum and in the anxiety spectrum)—such as for example cognitive-behavioral therapy (CBT). It also includes various neurofeedback and biofeedback treatments for various neuropsychiatric pathologies such as ADHD and pain syndromes. For some of the treatment interventions the monitoring may be based upon the Brain Engagement Index for enhanced attention, while for other interventions the monitoring may be based upon the comfort index to increase relaxation.

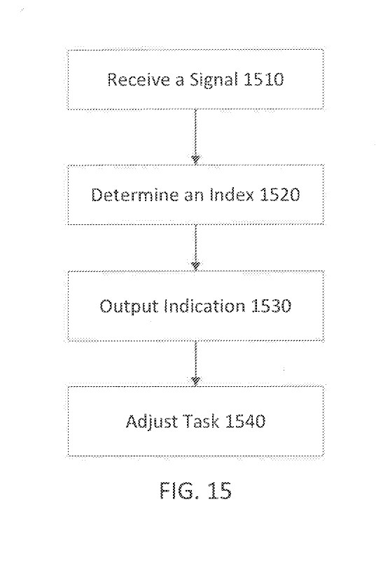

Consistent with disclosed embodiments, system 300 may be configured to perform a method of neural rehabilitation treatment management. As depicted in FIG. 15, this method may include the steps of receiving a signal in step 1510, determining an index in step 1520, outputting an indication in step 1530, and adjusting a task in step 1540. In step 1510, system 300 may be configured to receive a neurophysiological signal as described above. For example, signal processing unit 304 may be configured to receive the neurophysiological signal from signal source 302 and/or signal storage 308. In some embodiments, the neurophysiological signal may be received while the subject is engaged in an activity, such as a rehabilitation exercise or a training exercise. In step 1520, system 300 may be configured to determine an index as described with regards to any of FIG. 5 to FIG. 14. In some embodiments, signal processing unit 304 may be configured to determine a brain engagement index from the received signal as described with respect to FIGS. 9A and 9B. In some embodiments, step 1520 may also include determining a performance level of the rehabilitation exercise or training exercise.

In step 1530, system 300 may be configured to output an indication of a BEI value of the subject. This indication may comprise a visual, auditory, or tactile indication of the value of the brain engagement index. For example, a visual indication may be a value of the brain engagement index, or a symbolic representation of a value or range of values of the brain engagement index. For example, the symbolic representation may comprise a color or a shape. An auditory indication may comprise a sound representative of a value or range of values of the brain engagement index. A tactile indication may comprise a movement, pressure, or vibration representative of a value or range of values of the brain engagement index. In some embodiments, step 1530 may include outputting an indication of a performance level of the subject at the task.

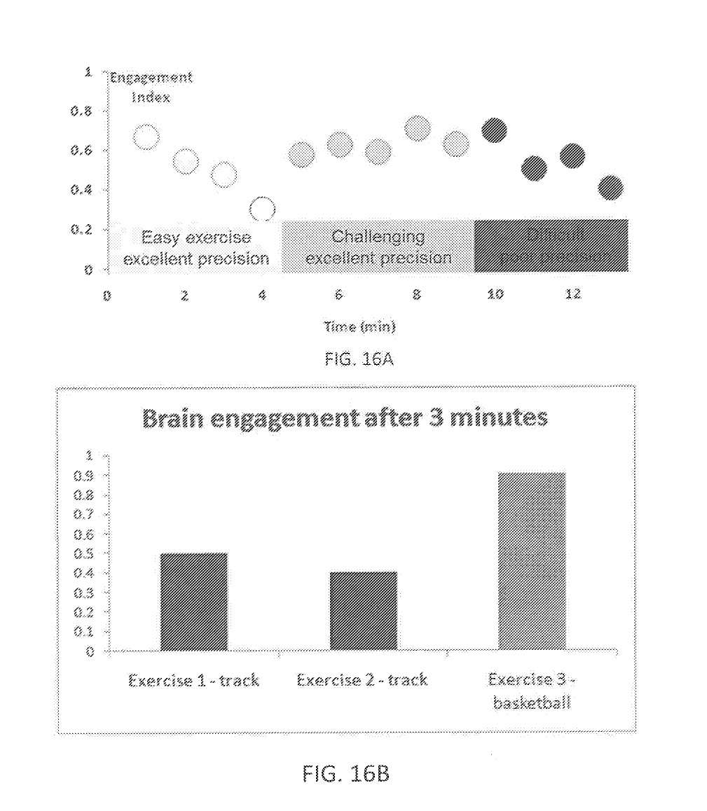

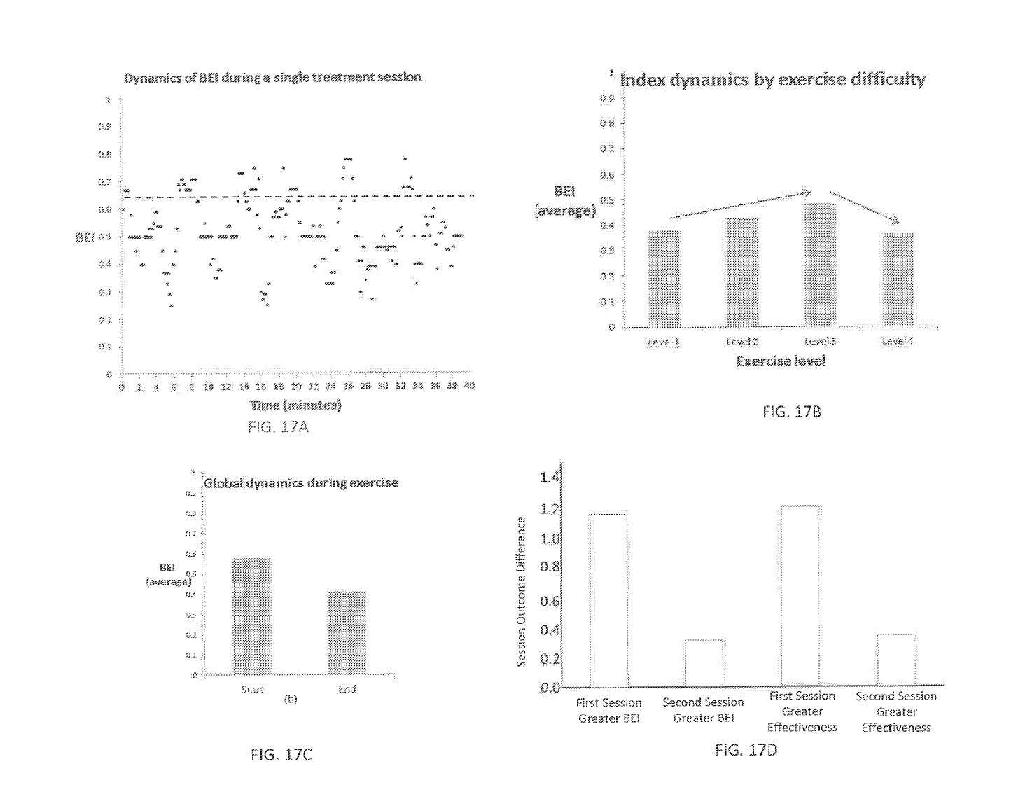

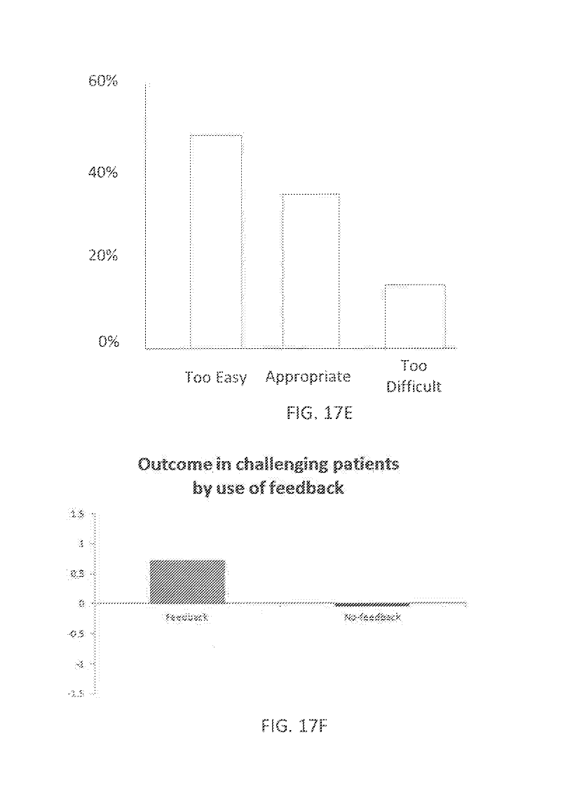

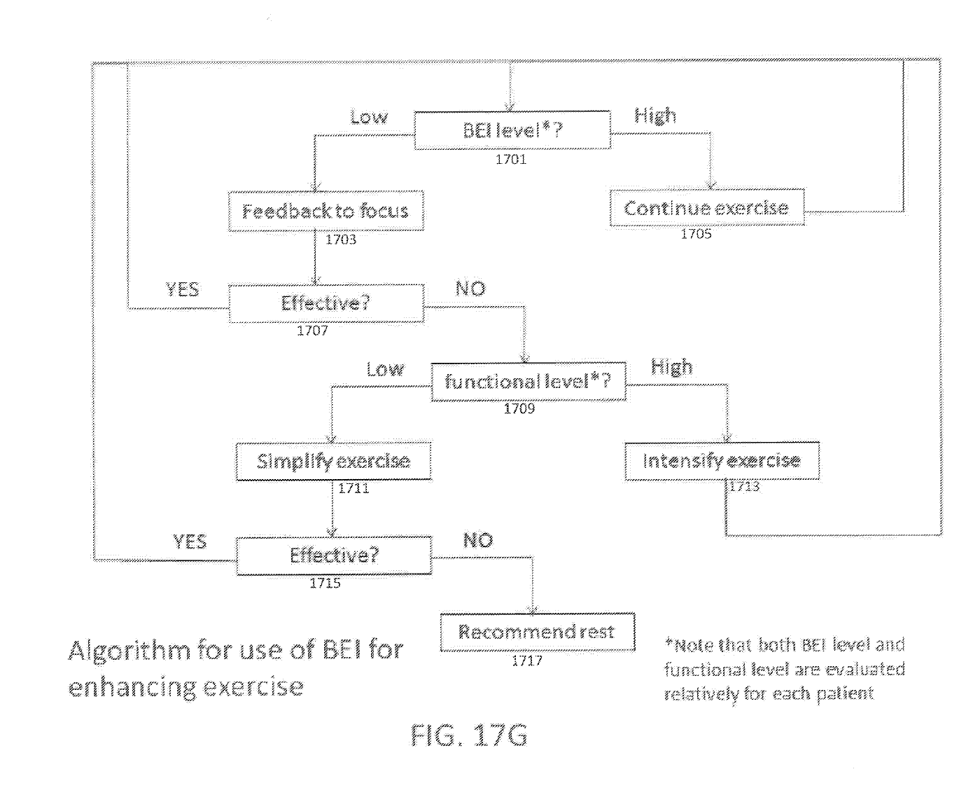

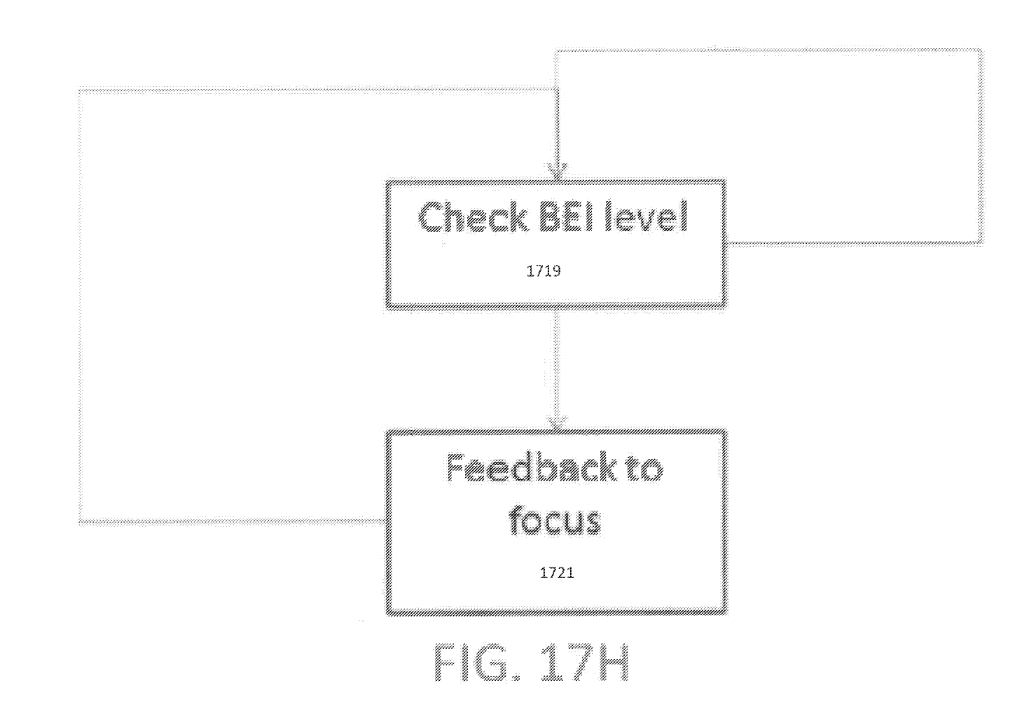

In step 1540, based on the output indication, the task may be adjusted. In some aspects, a difficulty of the task may be increased or decreased. For example, consistent with the results described below with respect to FIGS. 16A and 16B and FIGS. 17A to 17F, when BEI is below a threshold and performance exceeds a threshold, the difficulty of the rehabilitation exercise may be increased. As an additional example, when BEI is below a threshold and performance is below a threshold, the difficulty of the task may be decreased. In some embodiments, the adjustment of the difficulty of the task may be automatic. For example, system 300 may be configured to automatically adjust the difficulty of the task. In certain embodiments, the adjustment of the difficulty of the task may be manual. For example, the indication may be provided to prompt the subject or another person to adjust the difficulty of the task.

### Neural Rehabilitation Treatment Management Examples

FIGS. 16A and 16B show the effect of the different exercises on the subject's BEI over time. These figures show results from a study in which brain engagement was determined using signals received from a NeuroSky® EEG device. This device includes a forehead sensor and a reference sensor. Every 10 seconds, a normalized BEI value ranging from zero to one was calculated, as described above. As increased patient engagement may improve rehabilitation efficacy, low BEI may indicate poor rehabilitation efficacy while high BEI may indicate high rehabilitation efficacy. In some embodiments, the normalized BEI value should be greater than 0.7 during rehabilitation training.

As depicted in FIGS. 16A and 16B, a relationship may be observed between BEI index value and rehabilitation exercise characteristics. FIG. 16A depicts BEI values over time for a healthy subject instructed to perform a sit to stand rehabilitation exercise with different levels of difficulties, using a computerized rehabilitation device (LegTutor physical therapy produced by Meditouch LTD.). The rehabilitation exercise was performed at three levels of difficulty: easy, challenging, and difficult. As shown in FIG. 16A, the rehabilitation exercise was first performed at the easy level of difficulty. The subject easily performed well, and the measured BEI value of the subject decreased over time. The rehabilitation exercise was next performed at the challenging level of difficulty. Since good performance required the subject remain engaged in the exercise, the measured BEI value of the subject remained high. The subject could not achieve good performance of the exercise at the highest difficulty, and consequently experienced a decrease in brain engagement level. These results suggest that BEI values during rehabilitation exercise remain high, given an appropriate degree of rehabilitation exercise difficulty. In contrast, BEI values may decrease when the rehabilitation exercise is too easy or too difficult.

FIG. 16B depicts BEI values for a partially impaired subject, measured over three rehabilitation sessions totaling approximately 10 minutes. Between the first two sessions, a computerized rehabilitation device (the ArmTutor physical therapy product of Meditouch LTD.) automatically changed the rehabilitation exercise difficulty, based on the subject's performance. As shown in FIG. 16B, this did not result in effective levels of subject engagement, as demonstrated by low BEI values. In contrast, between the second and third sessions, rehabilitation exercise difficulty was changed taking both BEI values and performance into account. This resulted in an increase in subject BEI. After switching exercises, the patient experienced a significant reduction in impairment.

FIGS. 17A-17D depict the results of a study of 13 healthy subjects and 14 impaired subjects. The subjects performed a rehabilitation exercise using a MediTouch ArmTutor at four levels of difficultly. During performance of the rehabilitation exercise, a NeuroSky® MindWave™ system measured the subject's EEG, using a frontal electrode and a reference electrode on the earlobe. These measurements were transferred via wireless connection to a computer and the subject's BEI was processed online. Subjects underwent two treatment sessions: a “feedback session” and a “no-feedback session.” The order between the two treatment sessions (feedback and no-feedback) was randomized for each patient. Half of the patients started with the feedback session and half with the no-feedback session. BEI values were calculated online during both sessions. During the feedback session the treating physiotherapist responded to BEI decreases (of ˜0.2-0.3) by changing the rehabilitation exercise. For example, the treating physiotherapist changed the exercise (or exercise difficulty), paused the exercise, provided supportive exercises, and/or provided encouragement, in accordance with his clinical judgment. When changing the exercise level, the treating physiotherapist increased difficulty when BEI decreased and performance level was high, and reduced difficulty when BEI decreased and performance level was low.

Each treatment session was preceded and followed by 30 second evaluation periods, in which the motor function, which was the treatment goal, was tested and filmed. The evaluation films were evaluated by two physiotherapists, who were blinded to the use of BEI feedback. Each evaluating physiotherapist was asked to grade the functional change between pre-session and post-session evaluations on a [−3, +3] scale. Positive evaluation indicated improvement and negative evaluation indicated deterioration and the degree of improvement or deterioration is at the scale of 1 (minor) to 3 (major) accordingly. An evaluation of zero indicates no significant functional change between pre- and post-session evaluations.

During each treatment session an observer physiotherapist listed all the exercises employed and their start time and end time. The observer further evaluated each exercise according to its effectiveness on a one to three scale. An evaluation of one means that the exercise was too easy for the patient, three means that the exercise was too difficult and two means that the exercise was at an appropriate level for the subject.

Multiple variables were tracked during the study. For the rehabilitation task using the MediTouch ArmTutor, the study tracked the start-of-exercise BEI (the first BEI in each exercise), and the end-of-exercise BEI (the last BEI in the exercise). For the treatment sessions, the study tracked the session BEI, session effectiveness index, and session outcome index.

The session BEI was calculated as the portion of the treatment session in which BEI exceeds a threshold. As a non-limiting example, should the value of BEI exceed the threshold during 40 of 160 intervals in a treatment session then the session BEI would be 0.25. The session effectiveness index was calculated as the average of effectiveness evaluations for all the treatments in each session. The session outcome index was calculated as the average of session effect evaluations between the two blinded evaluators. This index was then compared between the feedback and no-feedback sessions or between the first and second sessions, regardless of which of them involved feedback.

FIG. 17A depicts the BEI dynamics during a treatment session. BEI values were calculated every 10 seconds using the previous 60 seconds of EEG data. FIG. 17B depicts the dynamics in end-of-exercise BEI. At each level the BEI index value may be averaged over all subjects. In this example, the index increases between the 1st level exercises and the 3rd level exercises (t-test paired, p<0.06). In this example, the index decrease from the 3rd level exercises to the 4th level exercises (t-test paired, p<0.001). FIG. 17C depicts the decrease in the index between the start-of-exercise BEI and the end-of-exercise BEI. The figure summarizes the data from both exercises at all 4 levels and for all 13 healthy subjects. It is possible to see the habituating effect of repetition (t-test paired, p<10−8).

FIG. 17D depicts the association between the session outcome index and both session BEI and session effectiveness index. The comparison may be between the session with high BEI and the session with low BEI for each subject, regardless of whether the high BEI session involved feedback or not. Similarly the comparison may be between the session with high effectiveness index and the session with low effectiveness index. The Y-axis presents the average outcome. It is possible to see that the outcome may be better when either session BEI (t-test paired, p<0.01) or session effectiveness (t-test paired, p≈0.03) index are larger. The differences for BEI and session effectiveness index seem similar in magnitude. For example, the session demonstrating the higher session BEI also demonstrated the higher session effectiveness index for subjects differing significantly in outcome between sessions. This further suggests that BEI may be an index for exercise effectiveness.

However, most sessions demonstrated a session effectiveness index outside the effective range. As shown in FIG. 17E, which depicts session effectiveness index for both sessions for all impaired subjects, the sessions were typically either too difficult or too easy. However, as shown in FIG. 17F, the clinical outcome improved more in sessions for which feedback was used for these impaired subjects (T-test paired, p≈0.04).

FIG. 17G and 17H depict flowcharts of an exemplary method for improving neural rehabilitation during rehabilitation exercises and/or ADLs, consistent with disclosed embodiments. As shown in FIG. 17G, the BEI values of the subject performing a rehabilitation exercise may be evaluated at step 1701. When BEI values are high, the rehabilitation exercise may continue without modification at step 1705. When BEI values are low or decreasing, the subject may be given feedback and instructed to focus on the rehabilitation exercise at step 1703. At step 1707, the effectiveness of the feedback may be evaluated. Should BEI values increase, the subject may continue performing the rehabilitation exercise (return to step 1701). Should BEI values remain low or continue to decrease, the subject's performance of the rehabilitation exercise may be evaluated at step 1709. When the subject's performance of the rehabilitation exercise is good, the difficulty of the rehabilitation exercise may be increased at step 1713, while continuing to monitor BEI values (return to step 1701). When the subject's performance of the rehabilitation exercise is poor, the difficulty of the rehabilitation exercise may be reduced at step 1711. At step 1715, the effectiveness of the reduction in difficulty may be evaluated. Should BEI values increase, the subject may continue performing the rehabilitation exercise (return to step 1701). Should BEI values remain low or decrease, the subject may be instructed to cease the rehabilitation exercises at step 1717.

In some embodiments, the relative change in BEI value and/or functional level may be evaluated. For example, the relative change in BEI value for a specific patient may be used to determine rehabilitation exercise difficulty for that patient. In some aspects, functional level during rehabilitation exercises may be evaluated by at least one of the caregiver and a computing device.

As shown in FIG. 17H, BEI values may be monitored during ADLs at step 1719. As long as BEI values remain high, system 300 will monitor and store activities. When BEI values are low or decreasing, the subject may be encouraged to focus at step 1721, and system 300 continues monitoring BEI values.

In this manner, the general method described above with regard to FIG. 15 may enable automatic or manual adjustment of rehabilitation exercises or ADLs to enhance rehabilitation efficacy.

### Training and Monitoring

As would be recognized by one of skill in the art, the systems and methods described above are not limited to neural rehabilitation treatment management and to treatment management of other neuropsychiatric disorders, as suggested above. Rather, these systems and methods may be generally applied to computer-based training applications as well as to non-computer-based training applications in non-clinical subjects. Consistent with disclosed embodiments, system 300 may be configured to measure BEI values and performance levels during training. Based on the measured BEI values and performance levels, the difficulty of the training may be adjusted. Non-limiting examples of computer-based training applications include athletic practice, scholastic instruction, and on-the-job training.

In some embodiments, a computer-based training system may be configured to interact with a subject performing a training task. In some embodiments, a difficulty level may be associated with the training task. For example, training tasks may be rated from low difficulty to high difficulty. As would be appreciated by one of skill in the art, the difficulty level may be an ordinal, interval, or ratio variable.

In some aspects, the computer-based training system may be configured to receive information concerning an interface presented to the subject. In certain aspects, the subject may interact with the interface, generating the information provided to the computer-based training system. In some aspects, the interface may include at least one of display elements and physical elements (e.g., microphones, gearshifts, throttles, steering wheels, tele-operation equipment, or similar physical elements).

The computer-based training system may be configured to receive at least one signal concerning the patient. The at least one signal may be received during or after performance of the training task by the subject. The at least one signal may be a physiological signal. For example, the signal may be an EEG or EMG signal. The computer-based training system may be configured to process the at least one signal to determine a mental state according to the systems and methods described above with regards to FIGS. 5 to 14.

The computer-based training system may also be configured to evaluate a performance level of the subject based on the received interface information. In some aspects, the computer-based training system may be configured to determine an absolute performance level. For example, the computer-based training system may be configured to determine whether a word is pronounced correctly, or the correct indicator in a GUI is selected. As an additional example, the computer-based training system may be configured to determine a relative performance level. For example, the computer-based training system may be configured to compare the performance of the subject to a performance criteria. In some aspects, the computer-based training system may be configured to generate this performance criteria. In certain aspects, the computer-based training system may be configured to receive this performance criteria. For example, the performance criteria may be received through interactions with a user of the computer-based training system. As an additional example, the performance criteria may be received from another computer system.

The computer-based training system may be configured to determine an adjustment to the difficulty of the task based on the performance level and the received at least one signal concerning the patient. For example, the computer-based training system may be configured to determine the adjustment according to the systems and methods described with regards to FIGS. 16A to 17H. In at least one embodiment, training environment parameters may be correlated to a marker or index indicative of subject mental state. As a non-limiting example, the computer-based training system may be communicatively connected to a vehicle braking and steering system. The computer-based training system may be configured to receive information concerning this vehicle braking and steering system. Information concerning braking or steering events may be correlated with the marker or index indicative of subject mental state to assess the subject's engagement or responsiveness to a driving situation.

The computer-based training system may be configured to provide an indication to adjust the difficulty level. For example, the computer-based training system may be configured to provide a control indication to adjust the difficulty level of the task. For example, the computer-based training system may be communicatively connected to a training device. As a non-limiting example, the computer-based training system may communicate with the training device over a network, serial connection, parallel connection, or bus. The computer-based training system may provide a command, instruction, or signal to the training device to adjust the difficulty level of the task. The training system may be configured to adjust the difficulty level of the task in response to the received command, instruction, or signal. As an additional non-limiting example, the computer-based training system may display the indication to prompt the subject, a practitioner, or another user to adjust the difficulty level of the task.

### Neuropsychiatric Treatment Management

The disclosed systems and methods may enable online, objective management of neuropsychiatric dysfunction. Neuropsychiatric dysfunction as used herein may refer to: mood problems such as depression; anxiety attacks; migraine attacks; epilepsy attacks; pain syndromes and psychoses and other DSM axis I psychopathologies; DSM axis II psychopathologies; pain; psychosomatic disorders; personal difficulty; stress-related disorder; mental load; ADD or ADHD. Optionally any other neuropsychiatric dysfunction may be included. Mental load refers to a mental construct that reflects the mental strain resulting from performing a task under specific environmental and operational conditions, coupled with the capability of the subject to respond to those demands.

System 300 may be configured to monitor a subject for neuropsychiatric dysfunction, consistent with disclosed embodiments. In some aspects, system 300 may be configured to monitor at least one of attentiveness markers, comfort markers, and focus event markers for a subject. In various aspects, system 300 may be configured to monitor at least one index, such as BEI, attention responsiveness index, and comfort index, as described above with regard to FIGS. 5 to 14. In some embodiments, system 300 may be configured to continuously measure EEG and determine such markers and indices while the subject carries out ADLs. Such determinations may enable system 300 to monitor mental overload in patients and in healthy subjects. In certain aspects, any of the markers or indices defined herein may be used for neuropsychiatric treatment management.

In some embodiments, system 300 may be configured for use with patients having at least one neuropsychiatric dysfunction. For example, system 300 may be configured to predict neuropsychiatric dysfunction, deterioration, and attacks; treatment effectiveness; and/or treatment outcome. As an additional example, system 300 may be configured to recommend interventions based on evaluation of neuropsychiatric dysfunction status. As a further example, system 300 may be configured to identify changes in effectiveness of current management of neuropsychiatric dysfunction. In various embodiments, these applications are based on at least one of mental state determination, as described with regards to FIGS. 5 to 14 above, a personal dysfunction log, a population dysfunction database, and an interventions log. For example, decrements in responsiveness can be used to predict mood problems and psychoses. Increments in responsiveness can be used to predict anxiety and migraine attacks. A pending attack can also be predicted by a significant decrement after an increment.

In some embodiments, system 300 may be configured to associate a change in marker values or index values with clinical events. In some aspects, system 300 may be configured to suggest interventions based on the change in marker value or index value. In various aspects, system 300 may be configured to evaluate the effectiveness of an intervention. For example system 300 may be configured to determine the change in a marker and/or index following an intervention. Interventions may include, without limitation, pharmaceutical; electromagnetic; psychological; behavioral; surgical; electrical, life style (e.g. exercise, nutrition, sleep), or similar interventions that would be recognized by one of skill in the art. In at least one embodiment, system 300 may be configured to use at least one of mental state determination, as described above with regards to FIGS. 5 to 14; additional health parameters received from at least one multi-sensor device (as a non-limiting example a health band or health watch); and pre-defined stored information related to changes in markers and/or indices associated with dysfunctional states.

As would be recognized by one of skill in the art, neural rehabilitation treatment management comprises a particular type of treatment management. The disclosed systems and methods are similarly applicable to other types of treatment management, such as CBT (cognitive behavioral therapy), transcranial magnetic stimulation (TMS), computer-based training, and management of ADD or ADHD.

As a non-limiting example, the disclosed systems and methods may be used for evaluation of subject ADD or ADHD status on a daily basis. In some aspects, such evaluations may be used to generate a recommended drug dosage intake. In certain aspects, this recommended dosage may depend on anticipated future subject activities. For example, the recommended drug dosage may be increased based on current mental state and anticipated future subject activities requiring attention, such as completing homework, studying, or other learning tasks.

In some embodiments, system 300 may be configured for online evaluation of subject ADD or ADHD status during treatment sessions (e.g., neuro-feedback sessions). Such online evaluation may be used to for task optimization. In some aspects, system 300 may be configured to fine-tune interventions based on evaluations of subject mental state as described above. For example, various interventions, such as electromagnetic interventions and behavioral interventions, may be adjusted based on indications provided by system 300.

In some embodiments, system 300 may be configured to support clinical trials. For example, system 300 may be configured to provide objective evidence of intervention effects. Similarly, system 300 may be configured to improve patient selection by identifying patients likely to benefit from treatment.

In various embodiments, system 300 may comprise a clinical or personal diagnostic tool. For example, system 300 may be configured for personal health management of neuropsychiatric dysfunctions (including home use). In some embodiments, system 300 may predict neuropsychiatric deterioration and provide intervention recommendations in advance of increasing neuropsychiatric dysfunction. The degree of advance notice may depend on the type of neuropsychiatric dysfunction. For example, such predictions provide weeks of advance notice for depression, days of advance notice for migraine attacks, and hours of advance notice for ADHD dysfunction.

### Mobile Mental State Tracker

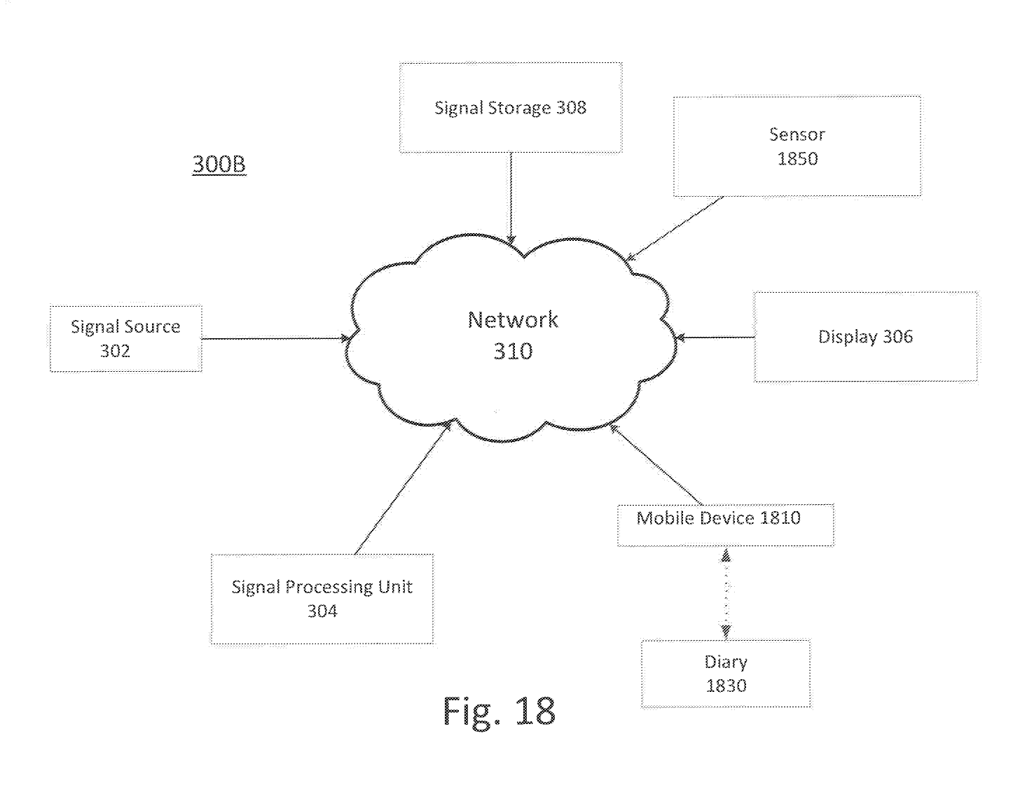

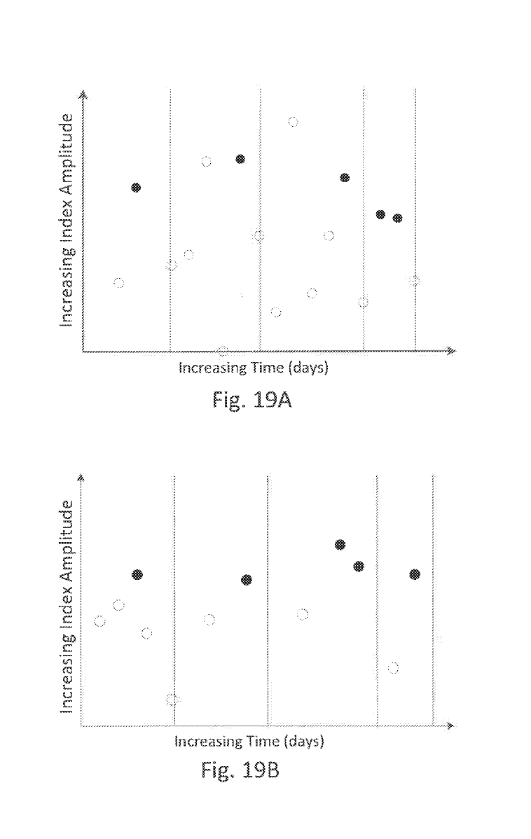

FIG. 18 depicts a schematic of an exemplary system 300B for mobile mental state tracking. In some embodiments, system 300 may be augmented to include mobile device 1810. Using mobile device 1810, system 300B may be configured to monitor a mental state of a subject. In some aspects, system 300B may be configured to generate diary 1830 of the subject's mental state. In various aspects, system 300B may be configured to use diary 1830 to determine behavioral adjustments for the subject. For example, system 300B may be configured to determine suitable times for undertaking activities based on historical personal date recorded in the mental state diary, such as suitable times for engaging in tasks that demand attention or focus. As an additional example, system 300B may be configured to identify behaviors resulting in improved focus, such as improved sleep and exercise habits.

Mobile device 1810 may include a fitness tracker or other wearable device, a smartphone, tablet, laptop, or similar device. In certain aspects, mobile device 1810 may be configured to operate diary 1830. In various embodiments, mobile device 1810 may be configured to operate diary 1830 to communicate diary information with another system that maintains diary 1830. For example, diary 1830 may be maintained on a separate cloud-based system. In some embodiments, diary 1830 may include at least one of a personal dysfunction log, population dysfunction database, and interventions log.

A personal dysfunction log may comprise a record of significant clinical events for an individual. A population dysfunction log may comprise a record of significant clinical events for a specific population. In some aspects, the specific population may comprise individuals characterized by a common or similar dysfunction. An interventions log may comprise a record of interventions as described above, related to a specific dysfunction.

In some embodiments, system 300B may comprise a processor and a computer-readable medium storing instructions that when executed by a processor cause the processor to perform operations. The operations may comprise a method for measuring mental states and may be performed at least in part on a mobile device (e.g., mobile device 1810). The operations may comprise receiving, on the mobile device, as a subject goes about daily activities, electrophysiological data from at least one pair of electrodes associated with the subject's head (e.g., signal source 302). The operations may further comprise determining from the electrophysiological data Brain Engagement Index. The operations may also comprise, based on the Brain Engagement Index, determining mental state measures for the subject as the subject goes about daily activities. The operations may additionally comprise generating a diary of the mental state measures over time (e.g., diary 1830). And the operations may further comprise outputting on the mobile device an indication of a mental state based on information stored in the diary. For example, the indication may be output on display 306

In some embodiments, the diary may further include a record of physical state measures concerning at least one of bodily functions, activity levels, and medication consumption. At least one of the physical state measures may be generated by a sensor other than an electrophysiological sensor. For example, system 300B may comprise sensor 1850. In some embodiments, sensor 1850 may comprise a component of mobile device 1810. In certain embodiments, sensor 1850 may comprise a component of a separate mobile device, such as fitness tracker or other wearable device, or an internal or external medical device (e.g. a pacemaker or an external ECG monitor), or a similar device. The operations may further include correlating the physical state measures with one or more of the mental state measures determined from the electrophysiological data. The mental state may be a future mental state, and the indication may relate to one or more of an attack (e.g. migraine, epilepsy) and a behavioral alteration. The behavioral alteration may include an increase or a decrease in at least one of anxiety, mania, depression, impulsivity, attentiveness, and hyperactivity.

As shown in FIGS. 19A and 19B, a diary enabling tracking of indices over time may allow for prediction of future mental or physiological states. FIG. 19A depicts the results of tracking a responsiveness index calculated using a brain engagement index as described above. The x-axis indicates time in days, while the y-axis indicates increasing index value. The vertical lines indicate days in which the subject suffered a migraine attack. Pre-attack days (1-2 days before migraine days) are shown in closed circles, while other days are shown as open circles. As may be seen from FIG. 19A, pre-attack days were associated with an increase in the index above a threshold. Similarly, FIG. 19B depicts the results of tracking a responsiveness index generated from event-related potentials as described above. The x-axis indicates time in days, while the y-axis indicates increasing index value. The vertical lines indicate days in which the subject suffered a migraine. Pre-attack days (1-2 days before migraine days) are shown in closed circles, while other days are shown as open circles. As in FIG. 19A, values of the responsiveness index greater than a threshold predict subsequent migraines.

### Mental Health Tracking Using EEG and Sensor Information

Consistent with disclosed embodiments, system 300 may be augmented to include sensor 2010 for monitoring physiological signals. Sensor 2010 may comprise one or more motion sensors, or physiological sensors for measuring signals such heart rate, body temperature, sweat chloride, respiration, blood oxygenation, skin galvanic response, or other such physiological signals. Sensor 2010 may be a component of a fitness tracker or other wearable device, or an internal or external medical device (e.g. a pacemaker or an external ECG monitor), or a similar device. In some embodiments, system 300 may be configured to monitoring EEG signals, temporally correlating those EEG signals with the signals from sensor 2010, and using the combined information to provide a personalized evaluation.

In some embodiments, system 300 may comprise at least one processor and a computer-readable medium storing instructions that when executed by the processor cause the processor to perform operations. The operations may include receiving electrophysiological signals from a head of a subject (e.g., from signal source 302). The operations may also include determining, from the electrophysiological signals, temporal sequences of amplitudes. The operations may further include receiving motion and/or physiological sensor signals from a sensor worn by the subject (e.g. sensor 2010), correlating the temporal sequences of amplitudes with the received motion sensor signals, and providing a personalized evaluation of the subject based on the correlation between the temporal sequences of amplitudes and motion sensor signals.

In some embodiments, the correlating may occur with respect to time. In certain embodiments, the physiological sensor may comprise at least one of an electrocardiogram, photoplethysmogram, galvanic skin response, bio-impedance, accelerometer, and skin temperature sensor. The physiological sensor signals may indicate an activity level of the subject, and the personalized evaluation may indicate a relationship between the activity level and the EEG signals. The operations may further comprise sharing the personalized evaluation of the subject in response to a sharing request received from the subject.

### EXAMPLE 1

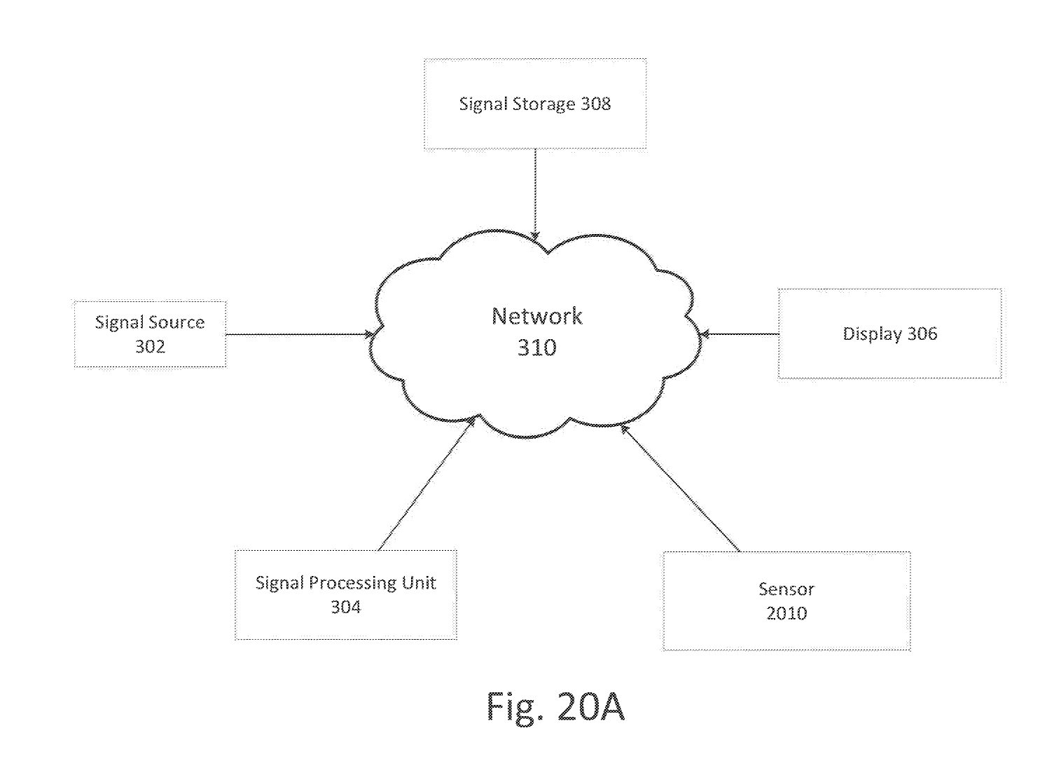

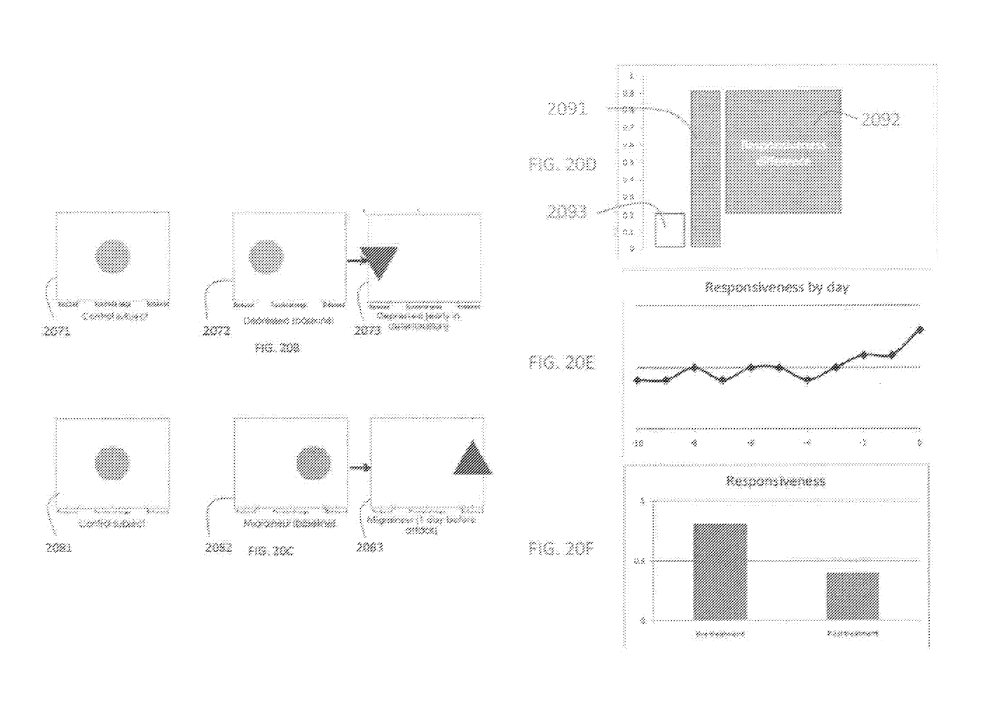

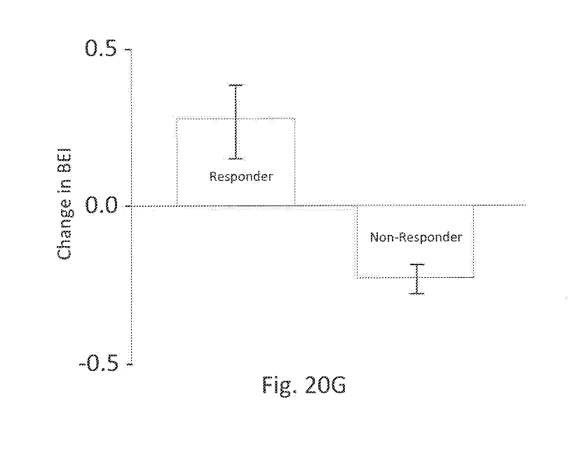

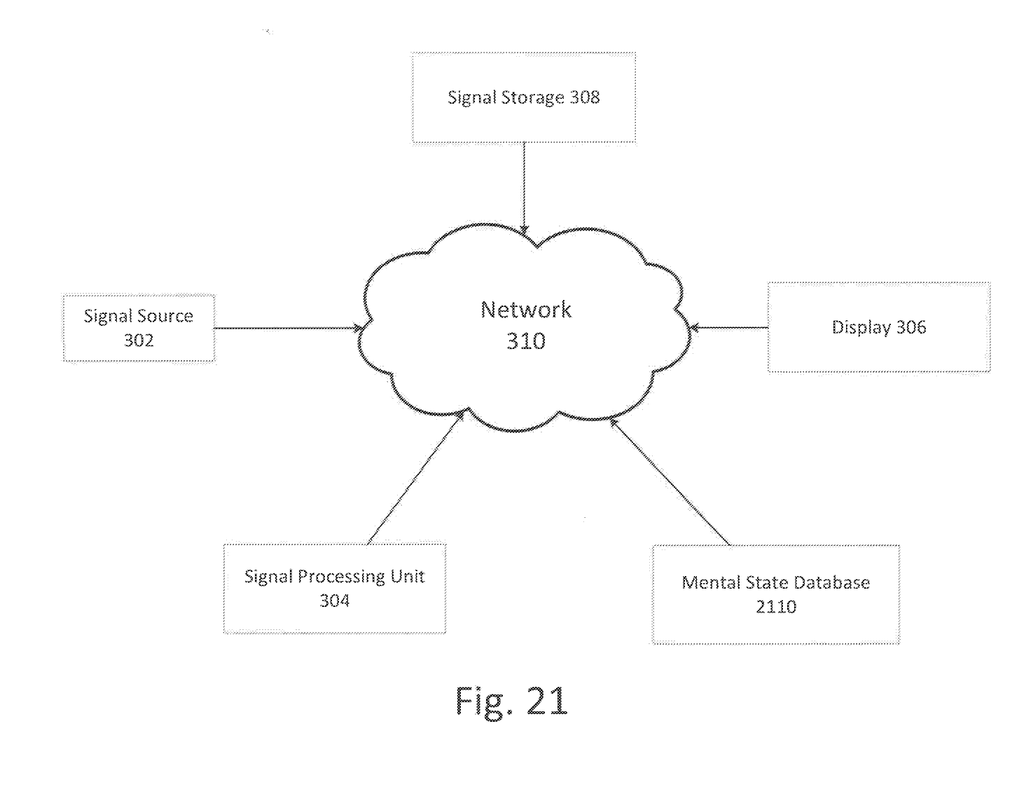

This example relates to a non-limiting, illustrative method for prediction of neuropsychiatric dynamics using the disclosed systems and methods. Reference is now made to FIG. 20B which shows evaluation of neuropsychiatric dysfunctions and prediction of deterioration/improvement according to at least one embodiment of the disclosed systems and methods.

FIG. 20B depicts evaluation and prediction of deterioration for a depressive subject, based on data provided in “Endogenous evoked potentials assessment in depression: a review,” by Nandrino, Massioui, and Everett, hereby incorporated by reference in its entirety. Box 2071 graphically illustrates a measure for a control subject previously determined to be non-depressive. This measure may be situated within a functional range (index value of ˜0.3-0.7) determined from population data, or from data specific to the individual. Box 2072 graphically illustrates a measure for a patient previously determined to be depressive, in a non-depressive state. The measure for this subject may be reduced, but remains within the functional range. Box 2073 graphically illustrates a measure for a patient previously determined to be depressive, during deterioration into a depressive state. This subject may exhibit a reduced measure below the functional range. Thus the method described above, using the novel markers and indices described above, can be used to predict deterioration of a depressive subject within a range of weeks.

### EXAMPLE 2

This example relates to a non-limiting, illustrative method for prediction of neuropsychiatric dynamics using the disclosed systems and methods. Reference is now made to FIG. 20C which shows evaluation of neuropsychiatric dysfunctions and prediction of deterioration according to at least one embodiment of the disclosed systems and methods.

FIG. 20C shows evaluation and prediction of deterioration for a migraine sufferer (migraineur), based on data provided in “Prediction of migraine attacks using a slow cortical potential, the contingent negative variation,” by Kropp and Gerber, hereby incorporated by reference in its entirety. Box 2081 graphically illustrates a measure for a non-migraineur control subject. Similarly, boxes 2082 and 2083 illustrate the measure for a migraineur in a healthy interictal state, and a migraineur one day prior to migraine attack respectively. The measure of the control subject in box 2081 may be in the middle of the functional range (0.3-0.7). The measure for the migraineur subject in an interictal state may be elevated within the functional range in box 2082. By contrast, the measure for the migraineur subject before the attack may be elevated above the normal range in box 2083. Thus the method described above, using the novel markers and indices described above, can be used to predict a migraine attack within a range of days. This relationship is also demonstrated above with respect to FIGS. 19A and 19B.

### EXAMPLE 3

This example relates to a non-limiting, illustrative method for evaluation of attention responsiveness using the disclosed systems and methods. Reference is now made to FIG. 20D which illustrates evaluation of attention responsiveness according to the disclosed systems and methods.

FIG. 20D shows the responsiveness of a subject to auditory stimulus. The responsiveness may be measured prior to the start of audio strip 2093 and then measured while hearing the audio strip 2091. The responsiveness difference 2092 may be calculated as the difference between the responsiveness measured prior to the start of audio strip and responsiveness measured while hearing the audio strip.

As shown in FIG. 20E, a subject's responsiveness may be measured daily using the method described above with respect to FIG. 20E. The daily deviations in attention responsiveness may be plotted to allow a medical practitioner to evaluate the dynamics of the subject. Alternatively or additionally, evaluation and recommendation of intervention may also be automatic. At least one of such evaluation and recommendation, whether or not generated automatically, could be population-based and/or patient-based.

### EXAMPLE 4

This example relates to a non-limiting, illustrative method for evaluation of attention responsiveness consistent with the disclosed systems and methods. Reference is now made to FIG. 20F, which illustrates evaluation of attention responsiveness consistent with the disclosed systems and methods.

As shown in FIG. 20F, measurements of attention responsiveness may be made using the disclosed systems and methods to evaluate treatment success. Attention responsiveness may be measured prior and post treatment and optionally during treatment. The resulting values may allow evaluation of treatment efficacy. For example, a reduction in responsiveness during treatment may provide an objective indication of treatment benefit.

### EXAMPLE 5

This example relates to a non-limiting, illustrative method for treatment evaluation and prediction consistent with the disclosed systems and methods. Reference is now made to FIG. 20G, which shows evaluation and prediction of pharmacological treatment efficacy in depression patients. This example shows the change in BEI values for six subjects diagnosed with depression. Brain engagement index was measured for each subject twice a week for two months after initiation of a new pharmacological treatment. The ordinate may be the measured change in BEI between initiation of the treatment and after two weeks of the treatment. Subjects were classified into responders and non-responders based on the long-term outcome of the pharmacological treatment. As shown in FIG. 20G, differences in BEI values between responders and non-responders appeared as early as two weeks. Over this time period, responders demonstrated an increase in BEI values, while non-responders demonstrated a decrease in BEI values. Accordingly, non-responding subjects may be quickly identified using BEI values, and saved the cost and inconvenience of ineffective pharmacological treatments for depression.

As shown in FIG. 20G, treatment responsiveness may be measured using the disclosed systems and methods. In some embodiments, the disclosed systems and methods may be used to predict the effectiveness of a new treatment regime. In various embodiments, the disclosed systems and methods may be used to evaluate treatment effectiveness for an existing treatment regime, for example by detecting increases or decreases in effectiveness.

Consistent with disclosed embodiments, a subject may initiate a new treatment regime addressing a neuropsychiatric dysfunction, as described above, such as depression. For example, untreated subjects may begin a treatment regime. As an additional example, subjects undergoing a first treatment regime may begin a second treatment regime. The second treatment regime may differ from the first treatment regime. For example the second treatment regime may differ from the first treatment regime in at least one of treatment modality, type, and parameter, as would be recognized by one of skill in the art. As a non-limiting example, treatment modalities may include medication, talk therapy, and medical interventions such as TMS, deep brain stimulation, electroshock therapy, or similar treatment modalities. As an additional non-limiting example, treatment type may include medication type, talk therapy type, medical intervention type, or similar treatment type. As a further non-limiting example, treatment parameters may include at least one of frequency, dosing, duration, intensity, and other relevant treatment parameters known to one of skill in the art. In certain embodiments, the subject may be monitored while maintaining an existing treatment regime.

Consistent with disclosed embodiments, BEI values may be repeatedly measured for subjects. For example, these repeated measurements may begin before initiation of the new treatment regime. As an additional example, these repeated measurements may begin upon or after initiation of the new treatment regime. As a further example, these repeated measurements may continue during monitoring of an existing treatment regime. In some aspects, the repeated measurements may be separated by intervals. For example, the repeated measurements may be separated by one or more minutes, hours, days, weeks, or months. The intervals separating measurements may not be constant. For example, a first two measurements in a sequence of repeated measurements may be separated by approximately an hour, and a second two measurements in the sequence of repeated measurements may be separated by a month. In some aspects, BEI values may be repeatedly measured for subjects approximately twice a week.

The overall duration of the repeated measurements for a subject may be less than one or more hours, days, weeks, or months. The overall duration of the repeated measurements may vary between subjects. In some aspects, the overall duration of the repeated measurements may be approximately two months.

Monitoring of treatment regime responsiveness may additionally depend on other measures. For example, in addition to the repeated measurements of BEI value, the envisioned systems and methods may include completion of questionnaires. Questionnaires and measurements may be completed at the same time. For example, an office visit by a subject may include completion of a questionnaire and measurement of BEI value. Alternatively or additionally, questionnaires and measurements may be completed at different times. For example, an office visit may include completion of a questionnaire or measurement of BEI value, but not both. The total number of completed questionnaires may be smaller, equal, or larger than the total number of measurements. As a non-limiting example for depression, the questionnaires may include at least one of the Hamilton Depression Rating Scale, Hamilton Anxiety Rating Scale , Visual Analogue Scale, and Clinical Global Impression.

As described above with regard to FIGS. 5 to 14, the BEI measurements may be determined from electrophysiological signals of the subject. In some aspects, the electrophysiological signals of the subject may be EEG signals. Consistent with disclosed embodiments, as described above, a BEI measurement may be determined from an interval of an electrophysiological signal. In certain aspects, the interval may be between 100 milliseconds and 5 minutes. In some aspects, the interval may be less than one second. In certain aspects, the interval may be less than 10 seconds. In various aspects, the interval may be less than 1 min. In some aspects, the interval may be less than 10 minutes.

As described above, with regards to FIG. 20G, an index may be calculated from the measured BEI values. For example, the index may comprise a change in the BEI values over time, as shown in FIG. 20G. Alternatively, the index may comprise a function of BEI values associated with a single treatment session, and the index may in turn be evaluated over time.

### Real Time Feedback on Mental Focus

In some embodiments, system 300 may be configured to use a downloadable application or “app” to measure a subject's current mental focus and provide real-time feedback. In this way, for example, if the subject loses focus on the task at hand, the subject may be prompted to become refocused.

In some embodiments, system 300 may comprise at least one processor and a computer-readable medium storing instructions. When executed by the at least one processor, the instructions may cause the at least one processor to perform operations. The operations may be performed at least in part on a mobile device. The operations may comprise receiving, on the mobile device, as a subject engages in an activity, electrophysiological measurements from at least one pair of electrodes associated with the subject's head. The operations may further comprise, based on the electrophysiological measurements received on the mobile device, determining an level of Brain Engagement Index (BEI) within a time window of less than two minutes. And the operations may comprise outputting, on the mobile device in real time, at least one indication of mental state based on the sequence of local maxima.

In some embodiments, the time window may be less than less than one minute, 30 seconds, 20 seconds, 15 seconds, 10 seconds, 5 seconds, or 1 second. In some aspects, the time window may be a trailing time window. In some aspects, the output may further include at least one of an audible, haptic, and visual signal. In various aspects the mental state may be focus on the activity.

In some embodiments, the activity may be rehabilitation therapy, the mental state may be an engagement level dependent on the length of the Brain Engagement Index, and the at least one indication may be output to prompt an adjustment of the rehabilitation therapy.

In various embodiments, the adjustment depends on a performance level of the patient at the rehabilitation therapy. In certain aspects, the adjustment includes modifying a difficulty of the rehabilitation therapy based on the engagement level and the performance level. The operations may further comprise determining a target mental state of the subject, identifying media content likely to move the mental state of the subject toward the target mental state, and presenting the identified media content to the subject. The operations may further include, after presenting the identified media content, adjusting the identified media content. Adjusting the identified media content may include at least one of increasing a volume of the identified media content, increasing a beat of the identified media content, adjusting a tone of the identified media content, and selecting new media content for presentation to the subject. The target mental state may depend on at least one of a time and the activity. The identification may depend on a known relationship between the identified media content and the target mental state. The identification may depend on a known relationship between the identified media content and a target mental state for a subject population. The identification may further depend on a similarity between the identified media content and previously provided media content, and an effect of the previously provided media content on a previous mental state of the subject.

In some embodiments, the activity may occurs in a training environment and the operations may further comprise receiving feedback information from a physical system with which the subject interacts during the activity. The operations may also comprise correlating the feedback information with the at least one indication of mental state. The operations may additionally comprise outputting an indication reflecting the correlation between the feedback information and the at least one indication of mental state. The training environment may include at least one of equipment on which the subject is training and a simulator on which the subject is training. The equipment includes at least one of an industrial machine, a computer, a vehicle, a health fitness machine, and a medical device. The feedback information may include at least one of braking, steering, and collision detection data. The feedback information may enable determination of subject errors.

In some embodiments, the operations may further include determining that the engagement level of the subject is less than a threshold value, and providing at least one of an audible, haptic, and visual signal to the subject. The indication may depict feedback information and engagement level over time.

### Neuro-Marketing & Content Selection and Evaluation

System 300 may be configured to use the disclosed markers and indices for at least one of neuro-marketing, content selection, and content evaluation, consistent with disclosed embodiments. In some embodiments, system 300 may be configured to use the disclosed markers and/or indices for at least one of the following: automatically recommending content; neuro-marketing and content evaluation; improving public reception of content (content encompassing, as a non-limiting example, media, films, messages, information, alerts, advertisements, and communications); measurement of media consumption; and content production.

In some embodiments, system 300 may be configured to use the disclosed markers and indices to evaluate the effect of content on a subject. System 300 may be configured to monitor the subject during content delivery, consistent with disclosed embodiments. System 300 may be configured to receive electrophysiological signals of the subject. System 300 may be configured to determine an indication of the mental state of the subject using the received electrophysiological signals. In some aspects, system 300 may be configured to determine the mental state indication using markers and indices, as disclosed above with regards to FIGS. 5 to 14. As a non-limiting example, system 300 may be configured to use focus event markers and the comfort index. As an additional non-limiting example, system 300 may be configured to monitor at least one of a tendency to action in response to content, comfort in response to content, and a focus effect in response to parts of content.

System 300 may be configured to select content for the subject, consistent with disclosed embodiments. In some aspects, system 300 may be configured to select this content using the mental state indication for the subject, and/or mental state indications for additional subjects. The mental state indications for additional subjects may have been previously determined. For example, the mental state indications for the additional subjects may have been determined while those additional subjects were exposed to the content, or to similar content.

System 300 may be configured with a recommendation system for content, consistent with disclosed embodiments. In some embodiments, the recommendation system may be configured to cross-reference a reaction of the subject to particular content with databases containing content preferences based on different methodologies. In some aspects, the recommendation system may use recommendation parameters based on the at least one of the disclosed markers and disclosed indices.

System 300 may be used for market research, consistent with disclosed embodiments. For example, system 300 may be configured to determine the effect of content on a subject in the context of a commercial environment. The commercial environment may comprise a supermarket, shopping center, movie theater, department store, or similar commercial, non-laboratory environment. In some aspects, system 300 may be augmented at least one additional sensor for recording subject information, such as an eye tracking system, a camera, an audiovisual recording system, or similar device. System 300 may be configured to determine correlations between user response and specific content exposure using data from the at least one additional sensor for recording subject information. In some aspects, system 300 may be augmented with at least one additional sensor to record environmental parameters, such as a thermometer for measuring temperature. System 300 may be configured to determine correlations between user response and environment conditions using data from the at least one additional sensor for recording environmental parameters.

System 300 may be used for recommending content, consistent with disclosed embodiments. In some embodiments, the content may be delivered in a car and at least one of the preference recommendations and volume are based on a combination of content enjoyment and BEI value. In some aspects the at least one of the preference recommendations and volume may be adapted to enhance driving safety. In some aspects, system 300 may be configured to monitor the user during the delivery of content (e.g., an audio track). System 300 may be configured to select additional content for the user based at least in part on the reactions of the subject to the previously provided content. In some embodiments, system 300 may be configured to analyze subject reaction data, store the subject reaction data in a database, and use the stored subject reaction data to provide additional content tailored to the mental state and preferences of the subject. In some aspects, subject reaction data may be cross-referenced with databases associating subject reactions with content preferences. In some aspects, these databases may be modified to account for data derived from preference databases.

In some embodiments, system 300 may be configured to select additional content based upon the following hierarchy: (1) population preference characteristics; followed by (2) semantic characteristics; followed by (3) physical characteristics. In some aspects, system 300 may be configured with a targeted effect. Whenever the provided content achieves the targeted effect, system 300 may be configured to select additional content considering similarity according to the above hierarchy: (1) population preference characteristics; followed by (2) semantic characteristics; followed by (3) physical characteristics. Optionally, whenever the provided content fails to achieve the targeted effect, system 300 may be configured to reexamine the provided content and determine new additional content.

Consistent with disclosed embodiments, system 300 may be configured to consider content similar based on user level data or aggregated population-level data. Content may be considered similar in a certain variable when the difference between them in that variable does not cross a defined threshold. In certain aspects, feedback for updating at least one of user and population rules may be implemented. In various aspects, data mining may be used to enhance the prediction rules.

In some embodiments, a similarity threshold crossing may be required in a certain variable. In certain aspect, it may be required that no significant change take place in other variables. The definition of this similarity threshold may be parametric. Such a definition may require a proximity rank for every two pieces in every variable. In some aspects, the proximity rank may be set at the user or population level. In some aspects, for scalar variables the proximity rank may be based on previous co-usage and also indirect co-usage (as a non-limiting example, a percentage in which each piece was used separately with other common pieces—which could be defined recursively for 1 intermediate piece, 2 intermediate pieces etc.). In various aspects, an order scale may be used for ordered variables. In certain aspects, as with prediction rules, the shift limitations could also be learned through data mining at one or more of the user or population level.

In some embodiments, during presentation of the content piece, the recommendation system may detect that the at least one of the disclosed markers and indices is trending away from at least one target value. For example, the user may not be enjoying the content piece. The recommendation system may then be configured to shift presentation to another content piece. Intermediate exit points may be defined in each piece to enable a smoother exit. In at least one embodiment, the prediction of the next possible piece and shift limitations could be similar to those presented above. The degree of the relevant parameters might be different for the case of shifting during content pieces—for example enabling greater shifts. In some aspects, the variables used and the degree of thresholds may be learned by data mining.

In certain embodiments, the step of recommending a content piece may be determined by computation of a marker by integrating the relevant activity according to the spatiotemporal criteria. In at least one embodiment, the integral of differences between EEG data derived from pairs of electrodes may be correlated with predetermined neurophysiological processes matching the integration, and further utilized in selection of a subsequent piece to be played to the user.

Optionally, in the case in which no alternative gives clear results to direct the content selection, the use of sequences of short parts of content and evaluation provides direction as to which piece attains the target. From there the selection and continuation may be done as disclosed above.

Optionally, the content may be interspersed with commercials. In at least one embodiment, the commercials may be selected according to information gathered from the response to content pieces by the evaluation of internal variables (as well as from some contribution of other variables). For example, by enhanced responsiveness to certain words during a song. In at least one embodiment, the response to the commercials themselves may be evaluated similarly for effect. Optionally, commercials may also be selected as music tracks based on the variables learned in a manner, similar to that presented above. The major difference may be in the starting target variable which has enhanced responsiveness. Optionally, machine learning could be utilized to improve the prediction rules with regard to commercials too, either at the user or population level. Optionally, feedback could also be introduced.

In yet another embodiment for offering personalized preference recommendations from an array of options, data indicating user satisfaction may be collected. In at least one embodiment, discrepancy between satisfaction and achievement of neurophysiological target goals means that target goals are not proper. In at least one embodiment, automatic correction of the target selection could be offered based on at least elementary data mining such as linear regression. If there is no such discrepancy, but dissatisfaction correlates with target variable(s) the problem may be addressed by tuning the variables set. Optionally, user scales are provided with basic recommendations of the internal variables.

The effectiveness of the disclosed subject matter for use in content selection and evaluation is illustrated in examples 7-9 below.

### EXAMPLE 7

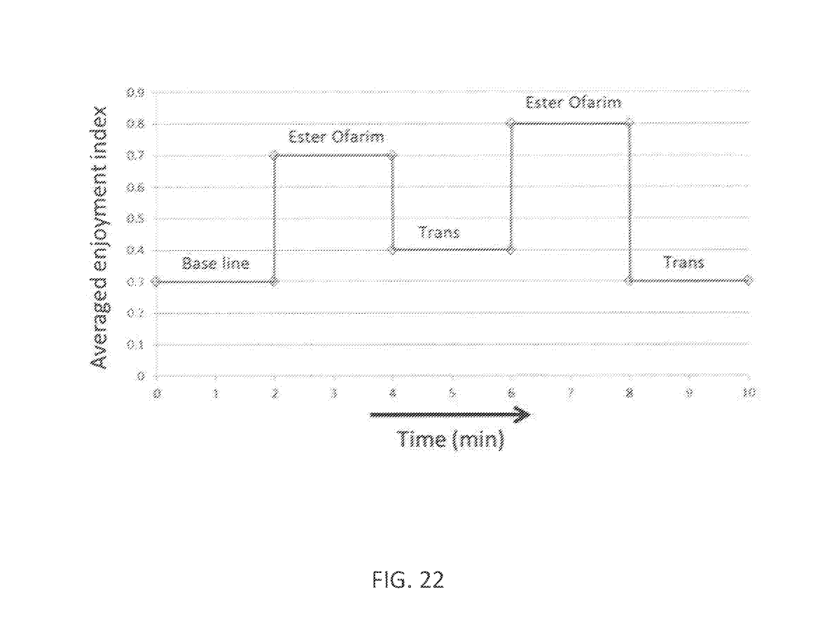

This Example relates to a non-limiting, illustrative method for media evaluation using embodiments consistent with the disclosed systems and methods. Reference is now made to FIG. 22-24 which show a subject's measured comfort index and focus events when exposed to different pieces of multimedia content consistent with disclosed embodiments.

As shown in FIG. 22, a subject was exposed to alternating audio stimulus of two music pieces for a duration of 2 minutes each. During the experiment, the subject's brain response was monitored by an EEG system using a Mindwave™ headset from NeuroSky®. The comfort index was calculated as described above during music listening and the index showed a marked increased when the subject's preferred music (Ester Ofarim) was played, as compared to a decrease in comfort during trance music. The comfort index was averaged for periods of two minutes.

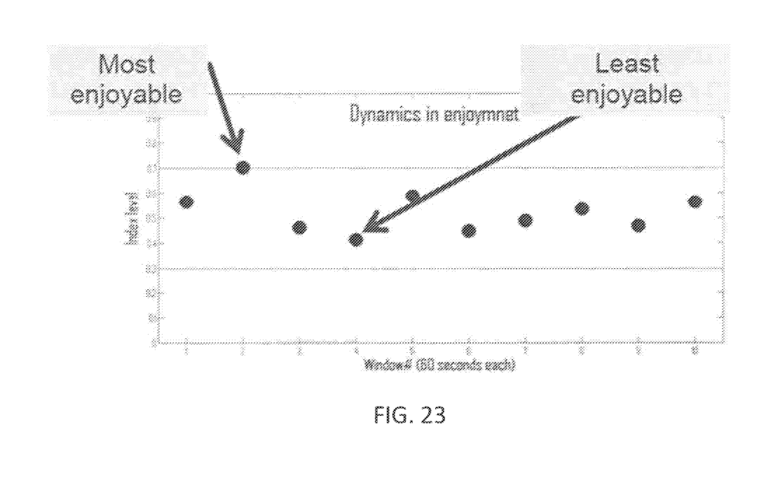

Similarly, in FIG. 23, ten commercial videos were played to a subject with the comfort index computed for periods of one minute allowing assessment of enjoyment corresponding to specific commercial or segment of the clip. Comfort values are relevant for durations of more than 30 seconds and correspond to an approximate 30-60 seconds delay after actual enjoyment occurred.

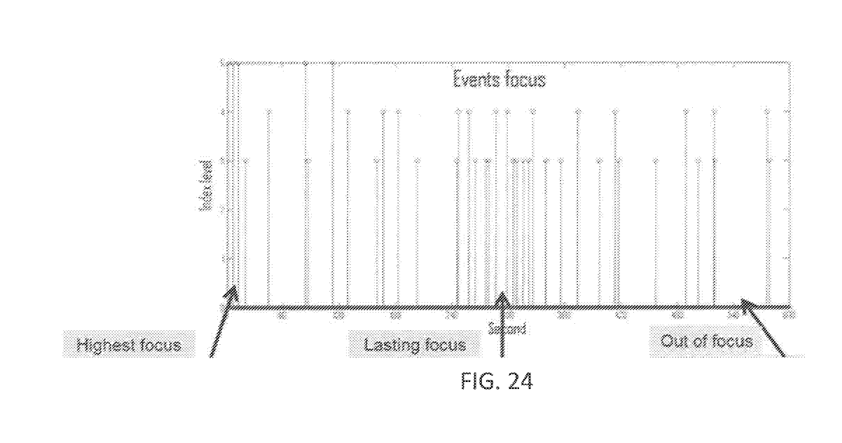

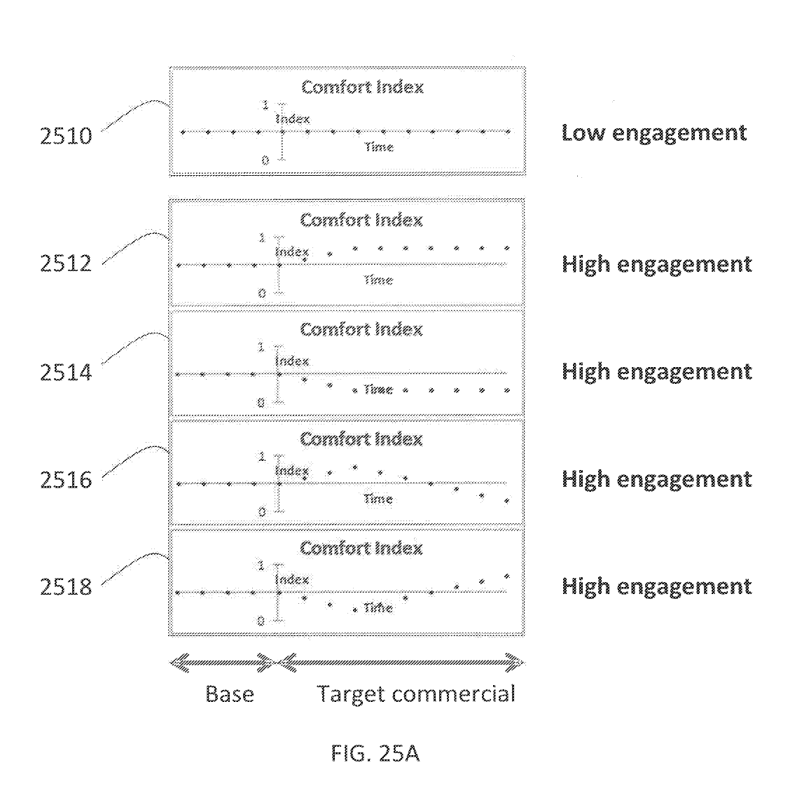

As shown in FIG. 24, focus events were calculated while exposed to audiovisual stimulus of the same ten commercial videos. Focus events are calculated as described above and were detected within one second from commencement.

Correlating EEG Signals in Real Time with a Presentation

In some embodiments, the disclosed systems may be configured to track changes in a subject's EEG in real time as the subject is exposed to a real-time presentation, and to determine, in real time, an impact that the presentation is having on the subject.

In some embodiments, system 300 may comprise at least one processor and a computer-readable medium storing instructions that when executed by at least one processor cause the at least one processor to perform operations. The operations may comprise monitoring, during a real-time presentation, a plurality of electrophysiological signals from a subject's head. The operations may further comprise identifying, in real time, at least one marker of Brain Engagement Index or of Comfort Index within at least one of the plurality of electrophysiological signals. The operations may additionally comprise determining a real-time mental state of the subject using the at least one sequence, correlating, in real time, the real-time mental state with a portion of the real-time presentation, and providing an indicator of an impact of the portion of the real-time presentation on the real-time mental state of the subject.

In some embodiments, the real-time mental state may be one or more of comfort, engagement, and attention. In certain aspects, the indicator may be provided within 5 seconds, 60 seconds, or 300 seconds of the portion of the real-time presentation. In various aspects, the real-time presentation includes traversal of a mall, store, or outdoor environment. In some aspects, the indication depicts the correlation between the real-time mental state and the portion of the real-time presentation.

### EXAMPLE 8

This Example relates to a non-limiting, illustrative method for neuro-marketing research using the disclosed systems and methods. The example illustrates how the disclosed markers and/or indices can provide the following information to researchers related to a piece of content: The overall degree of engagement evoked for the entire content piece, which strongly correlates with the subjects overall memory of the piece. Dynamics in emotion experienced by the subject while exposed to the content; and Indications of focus events at a ˜1 second resolution, which strongly correlates with memory of details within the content. This data provides a strong basis for the content owner or creator to assess emotional reaction—including negative reactions where these are desirable.

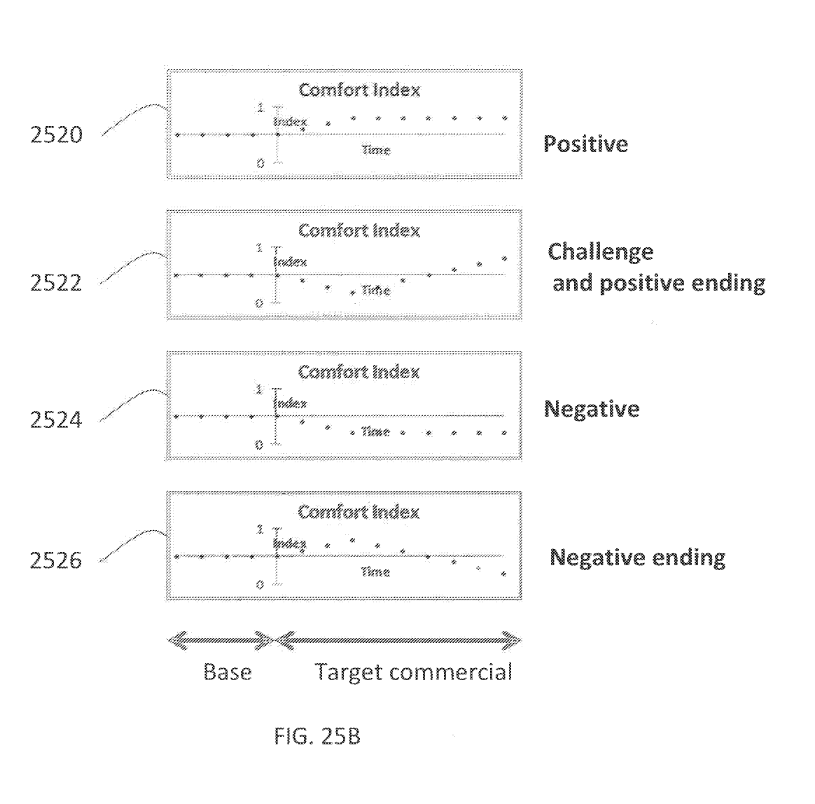

Reference is now made to FIG. 25A and 25B which show potential comfort index patterns and interpretations for a subject exposed to content consistent with disclosed embodiments.

FIG. 25A shows how the comfort index—derived as described above—can be used to assess how engaged the subject is in the chosen commercial. In each case the deviation from a baseline (before watching) is summed per commercial. The total deviation represents the engagement of the subject, which correlates strongly with the overall memory of the commercial. Graph 2510 shows practically no deviation and therefore a lack of subject engagement. By contrast, graphs 2512, 2514, 2516, and 2518 include multiple deviations and therefore high subject engagement.

FIG. 25B illustrates interpretation of the four “high-engagement” graphs of FIG. 25A where the direction of deviation represents emotion. Four possibilities of emotion dynamics are presented and, as noted above, the desired dynamics could change between commercials i.e.: a negative reaction might be the desired effect.

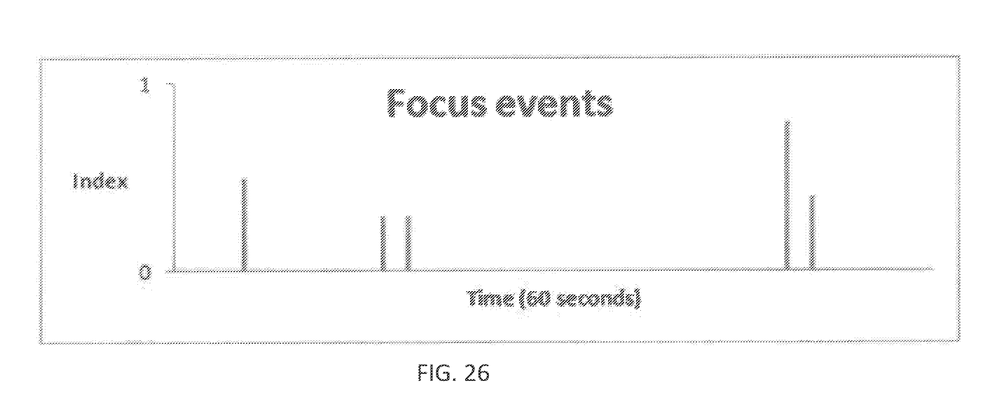

FIG. 26 illustrates determination of focus events at a ˜1 second resolution during the commercial. The identification of a focus event may correlate strongly with remembering the details presented at that time, for example, a brand name.

### EXAMPLE 9

This Example relates to a non-limiting, illustrative method for neuro-marketing research using the disclosed systems and methods. The example illustrates the use of the comfort index—as in Example 8—to allow assessment of a piece of content. In this case, two commercials were shown to a group of seven subjects and both the comfort index (for engagement and emotion) and focus events were analyzed. The professional selection criterion was expectancy of greater engagement in commercial B.

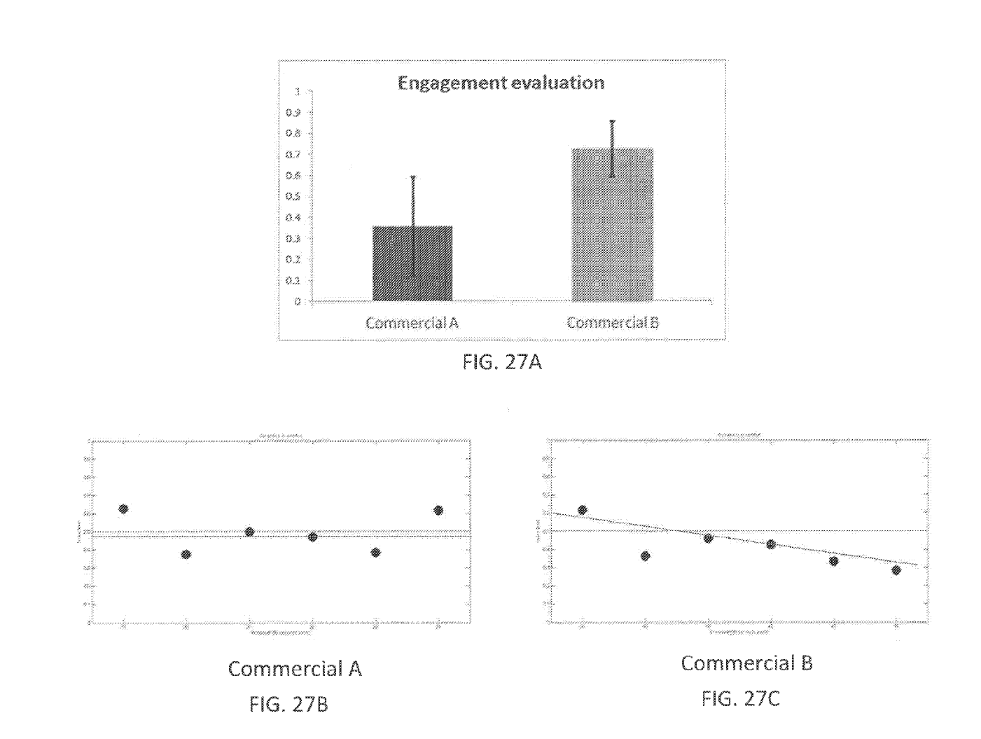

Reference is now made to FIGS. 27A-27C which show evaluation of user reaction to content consistent with disclosed embodiments.

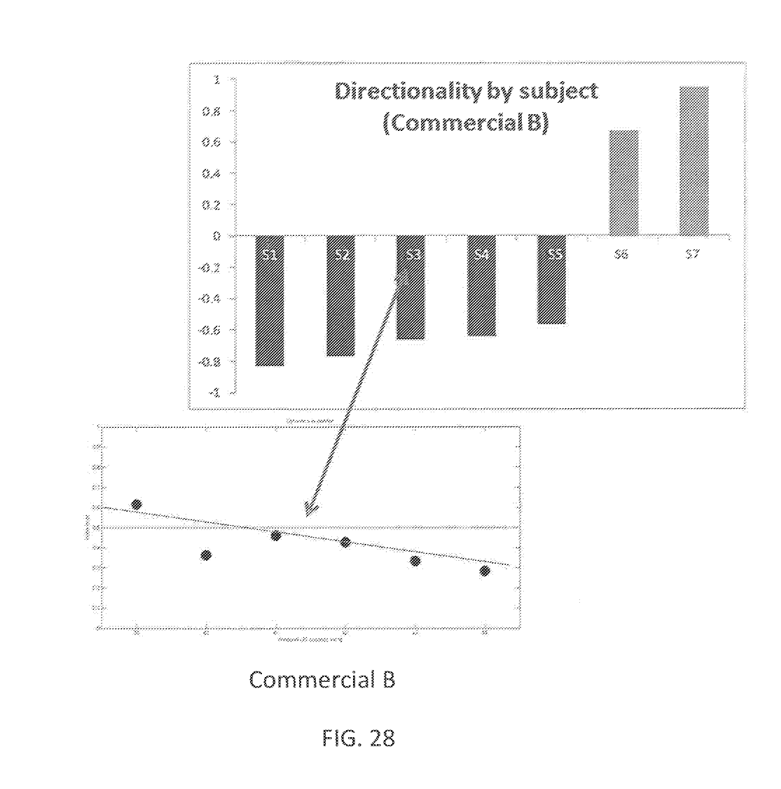

FIGS. 27A-27C shows the comfort index for one of the subjects for both commercials. As described above with reference to FIGS. 25A and 25B, the greater deviation related to commercial B indicating higher engagement by the subject. In FIG. 28, the evaluation of emotion by directionality showed commercial B had two opposite effects on the seven subjects. For most subjects it evoked discomfort but for two it evoked comfort effects. As noted above, both effects might lead to action and could therefore be desirable.

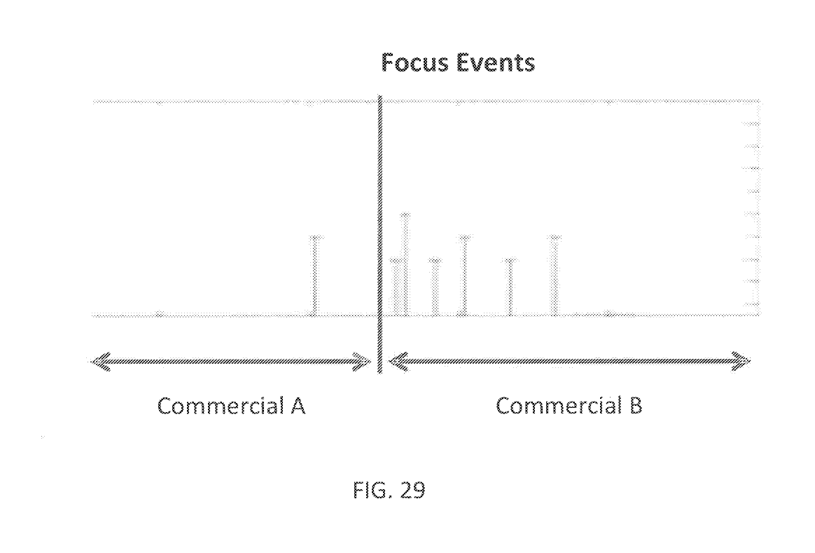

Finally, in FIG. 29, commercial B clearly has more focus events which as noted correlates strongly with remembering the details presented at that time.

### EXAMPLE 10

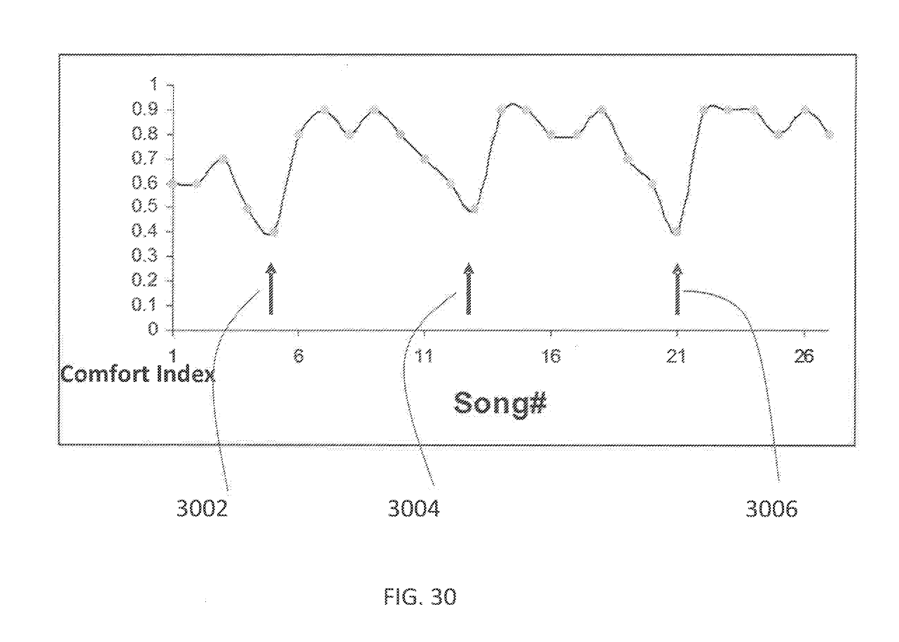

This Example relates to a non-limiting, illustrative method for media selection consistent with disclosed embodiments. Reference is now made to FIG. 30 which shows detection of comfort index in response to content played for use with a media selection algorithm to automatically select multimedia content consistent with disclosed embodiments.

As shown in FIG. 30, when the measured comfort index decreases as at points 3002, 3004, and 3006, the disclosed systems and methods will change the music selection and/or selection methodology. At each of points 3002, 3004, and 3006 the music database may be accessed to choose an appropriate piece of music while taking into account one or more of: Media characteristics such as artist, director, conductor, album, and/or genre; Audio characteristics such as beat-per-minute, loudness, bass, treble, tempo, left channel, and/or right channel; Environmental characteristics such as time, weather, and/or activity; Physiological characteristics such as heart rate, blood pressure, pupil dilation; and Population preference such as play counts, skip counts, ratings, and inclusion in playlists.

### Mental State Database for Recommendation Systems

In some embodiments, as shown in FIG. 21, system 300 may be augmented with mental state database 2110. In some embodiments, mental state databased 2110 may be configured to store associations between past EEG signals and past mental states. In certain aspects, mental state database 2110 may be configured to store associations between exposure to particular types of content, EEG signals, and past mental states. In some aspects, mental state database 2110 may be configured to store such associations for multiple users with similar traits or demographics. Using this stored information, system 300 may be configured to determine appropriate content for presentation to a user in order to achieve a desired effect.

In some embodiments, system 300 may comprise at least one processor and a computer-readable medium storing instructions that when executed by at least one processor cause the at least one processor to perform operations. The operations may comprise receiving, from a sensor worn on a head of a user (e.g., sensor 2010), electrophysiological signals. The operations may further comprise deriving first information relating to the mental state of the user from the received electrophysiological signals and storing the first information. The operations may comprise storing second information relating to initial content to which the user was exposed at a time of the mental state. The operations may also comprise receiving a prompt to present updated content to the user, wherein the prompt may be associated with causing a change in the mental state of the user, accessing third information reflective of mental states of other users, and determining, based on the first information, the second information and the third information, updated content to be presented to the user in an attempt to effect the change in the mental state. In some embodiments, at least one of the first information, second information, and third information may be stored in a database (e.g., database 2110).

In some embodiments, the prompt may be automatically generated based on a stored desired change in mental state. In some aspects, the prompt may be received from the user. In certain aspects, the third information may include mental state and associated updated content information for individuals with demographic similarities to the user. In various aspects, the third information may include mental state and associated updated content information for individuals with behavioral similarities to the user.

The foregoing detailed description of the envisioned subject matter, along with associated embodiments, has been presented for purposes of illustration only. It is not exhaustive and does not limit the envisioned subject matter to the precise form disclosed. Those skilled in the art will appreciate from the foregoing description that modifications and variations are possible in light of the above teachings or may be acquired from practicing the disclosed systems and methods. Likewise, the steps described need not be performed in the same sequence discussed or with the same degree of separation. Additionally, various steps may be omitted, repeated, or combined, as necessary, to achieve the same or similar objectives. Similarly, the systems described need not necessarily include all parts described in the embodiments, and may also include other parts not described in the embodiments. Accordingly, the envisioned subject matter is not limited to the above-described embodiments, but instead is defined by the appended claims in light of their full scope of equivalents.

What is claimed:

1. A non-transitory computer-readable medium storing instructions that when executed by at least one processor cause the at least one processor to perform operations comprising:
receiving at least one electrophysiological signal from at least one pair of electrodes on a head of a subject;
storing at least one threshold bio-electrical value;
comparing a plurality of portions of the at least one electrophysiological signal with the stored at least one threshold bio-electrical value;
determining, based on the comparing, identities of portions of the at least one electrophysiological signal that surpass the stored at least one threshold bio-electrical value;
determining the existence of a portion sequence among the portions of the at least one electrophysiological signal that surpass the stored at least one threshold bio-electrical value; and
outputting an indication of a mental state of the subject when it is determined that the portion sequence exists.

2. The non-transitory computer readable medium of claim 1, wherein the at least one pair of electrodes comprises a single pair of electrodes.

3. The non-transitory computer readable medium of claim 1, wherein the operations further comprise, prior to storing, determining the at least one threshold bio-electrical value by analyzing the at least one electrophysiological signal and identifying extremes in the at least one electrophysiological signal.

4. The non-transitory computer readable medium of claim 1, wherein determining the at least one first threshold bio-electrical value occurs by analyzing the at least one electrophysiological signal and identifying extremes in the at least one electrophysiological signal.

5. The non-transitory computer readable medium of claim 1, wherein the indication of the mental state indicates one or more of an attentiveness, focus, comfort, and responsiveness level of the subject.

6. The non-transitory computer readable medium of claim 1,
wherein the at least one received electrophysiological signal includes a first electrophysiological signal and a second electrophysiological signal, and
wherein the operations further comprise
determining a first sequence in the first electrophysiological signal and a second sequence in the second electrophysiological signal,
calculating a first measure of a mental state of the subject based on the first sequence and a second measure of the mental state of the subject based on the second sequence,
determining a mental condition of the subject based on the first measure and the second measure, and
wherein the indication of the mental state of the subject further comprises an indication of the mental condition.

7. The non-transitory computer readable medium of claim 1, wherein the mental condition relates to at least one of migraine, epilepsy, anxiety, ADD, ADHD, depression, psychosis, and dementia.

8. The non-transitory computer readable medium of claim 6, wherein the mental condition relates to at least one of migraine, epilepsy, anxiety, ADD, ADHD, depression, psychosis, and dementia.

9. The non-transitory computer readable medium of claim 6, wherein the operations further comprise updating a values sequence based on the first measure of mental state and the second measure of mental state, and wherein the indication of the mental condition is based on the values sequence.

10. The non-transitory computer readable medium of claim 9, wherein the indication of the mental condition is based on the values sequence and at least one of historical subject population data and historical subject data.

11. The non-transitory computer readable medium of claim 6, wherein the first electrophysiological signal occurs in an absence of an artificial stimulus, and wherein the second electrophysiological signal occurs, at least in part, in a presence of the artificial stimulus.

12. The non-transitory computer readable medium of claim 11, wherein the artificial stimulus is selected to assist in predicting, for a neuropsychiatric dysfunction, at least one of onset and severity, and wherein the indication of the mental condition concerns the prediction.

13. The non-transitory computer readable medium of claim 12, wherein the neuropsychiatric dysfunction comprises at least one of depression, anxiety, migraine, and ADD or ADHD.

14. A non-transitory computer-readable medium storing instructions that when executed by a processor cause the processor to perform operations on an electrophysiological signal obtained from at least two electrodes on a head of a subject, comprising:
determining a first value, when the electrophysiological signal has a first portion with a first duration,
at least one first sub-portion of the first portion with a second total duration, the at least one first sub-portion of the first portion satisfying first amplitude criteria,
at least one second sub-portion of the first portion with a third total duration, the at least one second sub-portion of the first portion satisfying the first amplitude criteria and second durational criteria, and
wherein the first value depends on the third total duration;
determining a second value that differs from the first value, when the electrophysiological signal has
a second portion with the first duration,
at least one first sub-portion of the second portion with the second total duration, the at least one first sub-portion of the second portion satisfying the first amplitude criteria,
at least one second sub-portion of the second portion with a fourth total duration, the at least one second sub-portion of the second portion satisfying the first amplitude criteria and the second durational criteria, the fourth total duration differing from the third total duration, and
wherein the second value depends on the fourth total duration; and
outputting an indication based on at least one of the first value and the second value.

15. The non-transitory computer-readable medium of claim 14, wherein the electrophysiological signal is a bandpass filtered version of an electrophysiological signal received from at least two electrodes on the head of the subject.

16. The non-transitory computer-readable medium of claim 14, wherein at least one sequence of peaks comprise the at least one first sub-portion of the first portion, the at least one sequence of peaks satisfying the first amplitude criteria.

17. The non-transitory computer-readable medium of claim 14, wherein at least one sequence of peaks comprise the at least one first sub-portion of the first portion, a function of the at least one sequence of peaks satisfying the first amplitude criteria.

18. The non-transitory computer-readable medium of claim 17, wherein the function comprises envelope detection.

19. The non-transitory computer-readable medium of claim 14, wherein the first value is monotonically nondecreasing with increasing third total duration.

20. The non-transitory computer-readable medium of claim 14, wherein the first value varies continuously with varying third total duration.

21. The non-transitory computer-readable medium of claim 14, wherein the second value indicates that the fourth total duration is zero.

22. The non-transitory computer-readable medium of claim 14, wherein the first value is independent of the first duration.

23. The non-transitory computer-readable medium of claim 14, wherein the first value depends on the second total duration and the third total duration.

24. The non-transitory computer-readable medium of claim 14, wherein the electrophysiological signal has been processed to remove each portion satisfying a noise criteria.
